SEQUENCE LISTING (10 sequences)

SEQ ID NO: 1 - DNA, 684 bp, Nicotiana tabacum
atgaatcccgctaatgcaaccttctctttctctgagcttgatttccttcaatcaatagaaaaccatcttctgaattatgattccgatttttctgaaattttttcgccgatgagttcaagtaacgcattgcctaatagtcctagctcaagttttggcagcttcccttcagcagaaaatagcttggatacctctctttgggatgaaaactttgaggaaacaatacaaaatctcgaagaaaagtccgagtccgaggaggaaacaaaggggcatgtcgtggcgcgtgagaaaaacgcgacacaagattggagacggtacataggagttaaacggcggccgtgggggacgttttcggcggagataagggacccggagagaagaggcgcgagattatggctaggaacttacgagaccccagaggacgcagcattggcttacgatcaagccgctttcaaaatccgcggctcgagagctcggctcaattttcctcacttaattggatcaaacattcctaagccggctagagttacagcgagacgtagccgtacgcgctcaccccagccatcgtcttcttcatgtacctcatcatcagaaaatgggacaagaaaaaggaaaatagatttgataaattccatagccaaagcaaaatttattcgtcatagctggaacctacaaatgttgctataa

SEQ ID NO: 2 - DNA, 378 bp, Artificial Sequence
  Description of Artificial Sequence Synthetic polynucleotide
ctaaccctgcacgttgtaatgaatttttaactattatattatatcgagttgcgccctccacttctagtctaaccctgcacgttgtaatgaatttttaactattatattatatcgagttgcgccctccacttctagtctaaccctgcacgttgtaatgaatttttaactattatattatatcgagttgcgccctccacttctagtctaaccctgcacgttgtaatgaatttttaactattatattatatcgagttgcgccctccacttctagaaattgtatttaaatgcatagatgtttattgggagtgtacagcaatctttcggaaaatacaaaccataatactttctcttcttcaatttgtttagtttaattttgaa

SEQ ID NO: 3 - DNA, 2046 bp, Nicotiana tabacum
atgactgattacagcttacccaccatgaatttgtggaatactagtggtactaccgatgacaacgttactatgatggaagcttttatgtcttctgatctcacttcattttgggctacttctaattctactgctgttgctgctgttacctctaattctaatcatattccagttaataccccaacggttcttcttccgtcttcttgtgcctctactgtcacagctgtggctgtcgatgcttcaaaatccatgtcttttttcaaccaagaaacccttcaacagcgtcttcaaacgctcattgatggtgctcgtgagacgtggacctatgccatcttttggcagtcatccgccgttgatttaacgagtccgtttgtgttgggctggggagatggttactacaaaggtgaagaagataaagccaataggaaattagctgtttcttctcctgcttatatagctgagcaagaacaccggaaaaaggttctccgggagctgaattcgttgatttccggcacgcaaaccggcactgatgatgccgtcgatgaagaagttaccgacactgaatggttcttccttatttccatgacccagtcgtttgttaacggaagtgggcttccgggtcaggccttatacaattccagccctatttgggtcgccggagcagagaaattggcagcttcccactgcgaacgggctcggcaggcccagggattcgggcttcagacgatggtttgtattccttcagcaaacggcgtggttgaattgggctccacggagttgattattcagagttctgatctcatgaacaaggttagagtattgtttaacttcaataatgatttgggctctggttcgtgggctgtgcaacccgagagcgatccgtccgctctttggctcactgatccatcgtctgcagctgtacaagtcaaagatttaaatacagttgaggcaaattcagttccatcaagtaatagtagtaagcaagttgtatttgataatgagaataatggtcacagttgtgataatcagcaacagcaccattctcggcaacaaacacaaggattttttacaagggagttgaacttttcagaattcgggtttgatggaagtagtaataataggaatgggaattcatcactttcttgcaagccagagtcgggggaaatcttgaattttggtgatagcactaagaaaagtgcaaatgggaacttattttccggtcagtcccattttggtgcaggggaggagaataagaagaagaaaaggtcacctgcttccagaggaagcaatgaagaaggaatgctttcatttgtttcaggtacaatcttgcctgcagcttctggtgcgatgaagtcaagtggatgtgtcggtgaagactcctctgatcattcggatcttgaggcctcagtggtgaaagaagctgaaagtagtagagttgtagaacccgaaaagaggccaaagaagcgaggaaggaagccagcaaatggacgtgaggaacctttgaatcacgtcgaagcagagaggcaaaggagagagaaattaaaccaaaggttctacgctttaagagctgttgttccgaatgtgtccaagatggacaaggcatcactgcttggagatgcaatttcatatattaatgagctgaagttgaagcttcaaactacagaaacagatagagaagacttgaagagccaaatagaagatttgaagaaagaattagatagtaaagactcaaggcgccctggtcctccaccaccaaatcaagatcacaagatgtctagccatactggaagcaagattgtagatgtggatatagatgttaagataattggatgggatgcgatgattcgtatacaatgtaataaaaagaaccatccagctgcaaggttaatggtagccctcaaggagttagatctagatgtgcaccatgccagtgtttcagtggtgaatgatttgatgatccaacaagccacagtgaaaatgggtagcagactttacacggaagagcaacttaggatagcattgacatccagagttgctgaaacacgctaa

SEQ ID NO: 4 - protein, 681 aa, Nicotiana tabacum
MTDYSLPTMNLWNTSGTTDDNVTMMEAFMSSDLTSFWATSNSTAVAAVTSNSNHIPVNTPTVLLPSSCASTVTAVAVDASKSMSFFNQETLQQRLQTLIDGARETWTYAIFWQSSAVDLTSPFVLGWGDGYYKGEEDKANRKLAVSSPAYIAEQEHRKKVLRELNSLISGTQTGTDDAVDEEVTDTEWFFLISMTQSFVNGSGLPGQALYNSSPIWVAGAEKLAASHCERARQAQGFGLQTMVCIPSANGVVELGSTELIIQSSDLMNKVRVLFNFNNDLGSGSWAVQPESDPSALWLTDPSSAAVQVKDLNTVEANSVPSSNSSKQVVFDNENNGHSCDNQQQHHSRQQTQGFFTRELNFSEFGFDGSSNNRNGNSSLSCKPESGEILNFGDSTKKSANGNLFSGQSHFGAGEENKKKKRSPASRGSNEEGMLSFVSGTILPAASGAMKSSGCVGEDSSDHSDLEASVVKEAESSRVVEPEKRPKKRGRKPANGREEPLNHVEAERQRREKLNQRFYALRAVVPNVSKMDKASLLGDAISYINELKLKLQTTETDREDLKSQIEDLKKELDSKDSRRPGPPPPNQDHKMSSHTGSKIVDVDIDVKIIGWDAMIRIQCNKKNHPAARLMVALKELDLDVHHASVSVVNDLMIQQATVKMGSRLYTEEQLRIALTSRVAETR

SEQ ID NO: 5 - DNA, 2148 bp, Nicotiana tabacum
cgcagacccctcttttcacccatttctctctctctctctctctctctctctatatatatatatatctttcacgccaccatatccaactgtttgtgctgggtttatggaatgactgattacagcttacccaccatgaatttgtggaatactagtggtactaccgatgacaacgtttctatgatggaatcttttatgtcttctgatctcacttcattttgggctacttctaattctactactgctgctgttacctctaattctaatcttattccagttaataccctaactgttcttcttccgtcttcttgtgcttctactgtcacagctgtggctgtcgatgcttcaaaatccatgtcttttttcaaccaagaaactcttcagcagcgtcttcaaaccctcattgatggtgctcgtgagacgtggacctatgccatcttttggcagtcatccgtcgttgatttatcgagtccgtttgtgttgggctggggagatggttactacaaaggtgaagaagataaagccaataggaaattagctgtttcttctcctgcttatattgctgagcaagaacaccgaaaaaaggttctccgggagctgaattcgttgatctccggcacgcaaaccggcactgatgatgccgtcgatgaagaagttaccgacactgaatggttcttccttatttccatgacccaatcgtttgttaacggaagtgggcttccgggtcaggccttatacaattccagccctatttgggtcgccggagcagagaaattggcagcttcccactgcgaacgggctcggcaggcccagggattcgggcttcagacgatggtttgtattccttcagcaaacggcgtggttgaattgggctccacggagttgataatccagagttgtgatctcatgaacaaggttagagtattgtttaacttcaataatgatttgggctctggttcgtgggctgtgcagcccgagagcgatccgtccgctctttggctcactgatccatcgtctgcagctgtagaagtccaagatttaaatacagttaaggcaaattcagttccatcaagtaatagtagtaagcaagttgtgtttgataatgagaataatggtcacagttctgataatcagcaacagcagcattctaagcatgaaacacaaggatttttcacaagggagttgaatttttcagaatttgggtttgatggaagtagtaataataggaatgggaattcatcactttcttgcaagccagagtcgggggaaatcttgaattttggtgatagtactaagaaaagtgcaaatgggaacttattttcgggtcagtcccattttggggcaggggaggagaataagaacaagaaaaggtcacctgcttccagaggaagcaatgaagaaggaatgctttcatttgtttcgggtacaatcttgcctgcagcttctggtgcgatgaagtcaagtggaggtgtaggtgaagactctgatcattcggatcttgaggcctcagtggtgaaagaagctgaaagtagtagagttgtagaacccgaaaagaggccaaagaagcgaggaaggaagccagcaaatggacgggaggaacctttgaatcacgtcgaagcagagaggcaaaggagagagaaattaaaccaaaggttctacgcattaagagctgttgttccgaatgtgtccaagatggacaaggcatcactgcttggagatgcaatttcatatattaatgagctgaagttgaagcttcaaaatacagaaacagatagagaagaattgaagagccaaatagaagatttaaagaaagaattagttagtaaagactcaaggcgccctggtcctccaccatcaaatcatgatcacaagatgtctagccatactggaagcaagattgtagacgtggatatagatgttaagataattggatgggatgcgatgattcgtatacaatgtaataaaaagaatcatccagctgcaaggttaatggtagccctcaaggagttagatctagatgtgcaccatgccagtgtttcagtggtgaacgatttgatgatccaacaagccactgtgaaaatgggtagcagactttacacggaagagcaacttaggatagcattgacatccagagttgctgaaacacgctaa

SEQ ID NO: 6 - protein, 679 aa, Nicotiana tabacum
MTDYSLPTMNLWNTSGTTDDNVSMMESFMSSDLTSFWATSNSTTAAVTSNSNLIPVNTLTVLLPSSCASTVTAVAVDASKSMSFFNQETLQQRLQTLIDGARETWTYAIFWQSSVVDLSSPFVLGWGDGYYKGEEDKANRKLAVSSPAYIAEQEHRKKVLRELNSLISGTQTGTDDAVDEEVTDTEWFFLISMTQSFVNGSGLPGQALYNSSPIWVAGAEKLAASHCERARQAQGFGLQTMVCIPSANGVVELGSTELIIQSCDLMNKVRVLFNFNNDLGSGSWAVQPESDPSALWLTDPSSAAVEVQDLNTVKANSVPSSNSSKQVVFDNENNGHSSDNQQQQHSKHETQGFFTRELNFSEFGFDGSSNNRNGNSSLSCKPESGEILNFGDSTKKSANGNLFSGQSHFGAGEENKNKKRSPASRGSNEEGMLSFVSGTILPAASGAMKSSGGVGEDSDHSDLEASVVKEAESSRVVEPEKRPKKRGRKPANGREEPLNHVEAERQRREKLNQRFYALRAVVPNVSKMDKASLLGDAISYINELKLKLQNTETDREELKSQIEDLKKELVSKDSRRPGPPPSNHDHKMSSHTGSKIVDVDIDVKIIGWDAMIRIQCNKKNHPAARLMVALKELDLDVHHASVSVVNDLMIQQATVKMGSRLYTEEQLRIALTSRVAETR

SEQ ID NO: 7 - DNA, 2241 bp, Nicotiana tabacum
cacacactctctccattttcactcactccttatcaccaaacaattcttgggtgtttgaatatatacccgaaataatttcctctctgtatcaagaatcaaacagatctgaattgatttgtctgtttttttttcttgattttgttatatggaatgacggattatagaataccaacgatgactaatatatggagcaatactacatccgatgataatatgatggaagcttttttatcttctgatccgtcgtcgttttggcccggaacaactactacaccaactccccggagttcagtttctccagcgccggcgccggtgacggggattgccggagacccattaaagtctatgccatatttcaaccaagagtcactgcaacagcgactccagactttaatcgatggggctcgcaaagggtggacgtatgccatattttggcaatcgtctgttgtggatttcgcgagcccctcggttttggggtggggagatgggtattataaaggtgaagaagataaaaataagcgtaaaacggcgtcgttttcgcctgactttatcacggaacaagcacaccggaaaaaggttctccgggagctgaattctttaatttccggcacacaaaccggtggtgaaaatgatgctgtagatgaagaagtaactgatactgaatggttttttctgatttccatgacacaatcgtttgttaacggaagcgggcttccgggcctggcgatgtatagttcaagcccgatttgggttactggaacagagagattagctgtttctcactgtgaacgggcccgacaggcccaaggtttcgggcttcagactattgtttgtattccttcagctaatggtgttgttgagctcgggtcaactgagttgatattccagactgctgatttaatgaacaaggttaaagttttgtttaattttaatattgatatgggtgcgactacgggctcaggatcgggctcatgtgctattcaggccgagcccgatccttcagccctttggctgactgatccggcttcttcagttgtggaagtcaaggattcgtcgaatacagttccttcaaggaataccagtaagcaacttgtgtttggaaatgagaattctgaaaatggtaatcaaaattctcagcaaacacaaggatttttcactagggagttgaatttttccgaatatggatttgatggaagtaatactcggtatggaaatgggaatgcgaattcttcgcgttcttgcaagcctgagtctggtgaaatcttgaattttggtgatagtactaagaggagtgcttgcagtgcaaatgggagcttgttttcgggccaatcacagttcgggcccgggcctgcggaggagaacaagaacaagaacaagaaaaggtcacctgcatcaagaggaagcaacgatgaaggaatcctttcatttgtttcgggtgtgattttgccaagttcaaacacggggaagtccggtggaggtggcgattcggatcaatcagatctcgaggcttcggtggtgaaggaggcggatagtagtagagttgtagaccccgagaagaagccgaggaaacgagggaggaaaccggctaacgggagagaggagccattgaatcatgtggaggcagagagacaaaggagggagaaattgaatcaaagattctatgcacttagagctgttgtaccaaatgtgtcaaaaatggataaagcatcacttcttggtgatgcaattgcatttatcaatgagttgaaatcaaaggttcagaattctgactcagataaagaggacttgaggaaccaaatcgaatctttaaggaatgaattagccaacaagggatcaaactataccggtcctcccccgtcaaatcaagaactcaagattgtagatatggacatcgacgttaaggtgatcggatgggatgctatgattcgtatacaatctaataaaaagaaccatccagccgcgaggttaatgaccgctctcatggaattggacttagatgtgcaccatgctagtgtttcagttgtcaacgagttgatgatccaacaagcgactgtgaaaatgggaagccggctttacacgcaagaacaacttcggatatcattgacatccagaattgctgaatcgcgatgaagagaaatacagtaaatggaaattatcatagtgagctctgaataatgttatctttcattgagctattttaagagaatttctcctaaaaaaaaaaaaaaaaaaaaaaaaaaa

SEQ ID NO: 8 - protein, 659 aa, Nicotiana tabacum
MTDYRIPTMTNIWSNTTSDDNMMEAFLSSDPSSFWPGTTTTPTPRSSVSPAPAPVTGIAGDPLKSMPYFNQESLQQRLQTLIDGARKGWTYAIFWQSSVVDFASPSVLGWGDGYYKGEEDKNKRKTASFSPDFITEQAHRKKVLRELNSLISGTQTGGENDAVDEEVTDTEWFFLISMTQSFVNGSGLPGLAMYSSSPIWVTGTERLAVSHCERARQAQGFGLQTIVCIPSANGVVELGSTELIFQTADLMNKVKVLFNFNIDMGATTGSGSGSCAIQAEPDPSALWLTDPASSVVEVKDSSNTVPSRNTSKQLVFGNENSENVNQNSQQTQGFFTRELNFSEYGFDGSNTRYGNGNANSSRSCKPESGEILNFGDSTKRSACSANGSLFSGQSQFGPGPAEENKNKNKKRSPASRGSNDEGILSFVSGVILPSSNTGKSGGGGDSDQSDLEASVVKEADSSRVVDPEKKPRKRGRKPANGREEPLNHVEAERQRREKLNQRFYALRAVVPNVSKMDKASLLGDAIAFINELKSKVQNSDSDKEDLRNQIESLRNELANKGSNYTGPPPSNQELKIVDMDIDVKVIGWDAMIRIQSNKKNHPAARLMTALMELDLDVHHASVSVVNELMIQQATVKMGSRLYTQEQLRISLTSRIAESR

SEQ ID NO: 9 - DNA, 2377 bp, Nicotiana tabacum
gtaacaaaccctctccattttcactcactccaaaaaactttcctctctattttttctctctgtatcaagaatcaaacagatctgaattgatttgggagttttttttcttcttgtttttgttatatggaatgacggactatagaataccaacgatgactaatatatggagcaatacaacatccgacgataacatgatggaagcttttttatcttctgatccgtcgtcgttttgggccggaacaaatacaccaactccacggagttcagtttctccggcgccggcgccggtgacggggattgccggagacccattaaagtcgatgccgtatttcaaccaagagtcgctgcaacagcgactccagacgttaatcgacggggctcgcgaagcgtggacttacgccatattctggcaatcgtctgttgtggatttcgtgagcccctcggtgttggggtggggagatggatattataaaggagaagaagacaagaataagcgtaaaacggcggcgttttcgcctgattttattacggagcaagaacaccggaaaaaagttctccgggagctgaattctttaatttccggcacacaaactggtggtgaaaatgatgctgtagatgaagaagtaacggatactgaatggttttttctgatttcaatgactcaatcgtttgttaacggaagcgggcttccgggcctggctatgtacagctcaagcccgatttgggttactggaagagaaagattagctgcttctcactgtgaacgggcccgacaggcccaaggtttcgggcttcagactatggtttgtattccttcagctaatggtgttgttgagctcgggtcaactgagttgatattccagagcgctgatttaatgaacaaggttaaaatcttgtttgattttaatattgatatgggcgcgactacgggctcaggttcgggctcatgtgctattcaggctgagcccgatccttcaaccctttggcttacggatccaccttcctcagttgtggaagtcaaggattcgtcgaatacagttccttcaagtaatagtagtaagcaacttgtgtttggaaatgagaattctgaaaatgttaatcaaaattctcagcaaacacaaggatttttcactagggagttgaatttttccgaatatggatttgatggaagtaatactaggagtggaaatgggaatgtgaattcttcgcgttcttgcaagcctagaaatgcttcaagtgcaaatgggagcttgttttcgggccaatcgcagttcggtcccgggcctgcggaggagaacaagaacaagaacaagaaaaggtcacctgcatcaagaggaagcaatgaagaaggaatgctttcatttgtttcgggtgtgatcttgccaagttcaaacacggggaagtccggtggaggtggcgattcggatcattcagatctcgaggcttcggtggtgaaggaggcggatagtagtagagttgtagaccccgagaagaggccgaggaaacgaggaaggaaaccggctaacgggagagaggagccattgaatcatgtggaggcagagaggcaaaggagggagaaattgaatcaaagattctatgcacttagagctgttgtaccaaatgtgtcaaaaatggataaagcatcacttcttggtgatgcaattgcatttatcaatgagttgaaatcaaaggttcagaattctgactcagataaagatgagttgaggaaccaaattgaatctttaaggaatgaattagccaacaagggatcaaactataccggtcctccaccgccaaatcaagatctcaagattgtagatatggatatcgacgttaaagtcatcggatgggatgctatgattcgtatacaatctaataaaaagaaccatccagccgcgaggttaatggccgctctcatggaattggacttagatgtgcaccatgctagtgtttcagttgtcaacgagttgatgatccaacaagcgacagtgaaaatggggagccggctttacacgcaagagcagcttcggatatcattgacatccagaattgctgaatcgcgatgaagagaaatacagtaaatggaaattattagtgagctctgaataatgttatctttcattgagctattttaagagaatttctcctatagttagatcttgagattaaggctacttaaaagtggaaagttgattgagctttcctcttagttttttgggtatttttcaacttttatatctagtttgttttccacattttctgtacatataatgtgaaaccaatactagatctcaagatctggtttttagttctgtaattagaaataaatatgcagcttcatctttttctgttaaaaaaaaaaaaaaaaaaaaaaaaa

SEQ ID NO: 10 - protein, 658 aa, Nicotiana tabacum
MTDYRIPTMTNIWSNTTSDDNMMEAFLSSDPSSFWAGTNTPTPRSSVSPAPAPVTGIAGDPLKSMPYFNQESLQQRLQTLIDGAREAWTYAIFWQSSVVDFVSPSVLGWGDGYYKGEEDKNKRKTAAFSPDFITEQEHRKKVLRELNSLISGTQTGGENDAVDEEVTDTEWFFLISMTQSFVNGSGLPGLAMYSSSPIWVTGRERLAASHCERARQAQGFGLQTMVCIPSANGVVELGSTELIFQSADLMNKVKILFDFNIDMGATTGSGSGSCAIQAEPDPSTLWLTDPPSSVVEVKDSSNTVPSSNSSKQLVFGNENSENVNQNSQQTQGFFTRELNFSEYGFDGSNTRSGNGNVNSSRSCKPESGEILNFGDSTKRNASSANGSLFSGQSQFGPGPAEENKNKNKKRSPASRGSNEEGMLSFVSGVILPSSNTGKSGGGGDSDHSDLEASVVKEADSSRVVDPEKRPRKRGRKPANGREEPLNHVEAERQRREKLNQRFYALRAVVPNVSKMDKASLLGDAIAFINELKSKVQNSDSDKDELRNQIESLRNELANKGSNYTGPPPPNQDLKIVDMDIDVKVIGWDAMIRIQSNKKNHPAARLMAALMELDLDVHHASVSVVNELMIQQATVKMGSRLYTQEQLRISLTSRIAESR

(19) United States
(12) Patent Application Publication
(10) Pub. No.: US 2022/0275387 A1
(43) Pub. Date: Sep. 1, 2022

(54) TRANSCRIPTION FACTOR NtERF221 AND METHODS OF USING THE SAME

(71) Applicants: University of Virginia Patent Foundation, Charlottesville, VA (US); 22nd Century Limited, LLC, Williamsville, NY (US)

(72) Inventors: Michael P. TIMKO, Charlottesville, VA (US); Hai Liu, Charlottesville, VA (US)

(73) Assignees: University of Virginia Patent Foundation, Charlottesville, VA (US); 22nd Century Limited, LLC, Williamsville, NY (US)

(21) Appl. No.: 17/632,652

(22) PCT Filed: Aug. 4, 2020
(86) PCT No.: PCT/US20/44831

§ 371 Date: Feb. 3, 2022

Related U.S. Application Data
(60) Provisional application No. 62/882,860, filed on Aug. 5, 2019.

(51) Int. Cl. C12N 15/82

(52) U.S. Cl. CPC C12N 15/8243

(57) ABSTRACT

The present technology provides transcription factors for modifying plant metabolism and nucleic acid molecules that encode such transcription factors. Also provide are methods of using these nucleic acids to modulate alkaloid production in plants and for producing plant and plant cells having altered alkaloid content. Disclosed herein are methods and compositions for modulating nicotine biosynthesis in plants.

### CROSS-REFERENCE TO RELATED APPLICATION

The present application claims priority to U.S. Provisional Patent Application No. 62/882,860, filed on Aug. 5, 2019, the contents of which are hereby incorporated by reference in their entirety.

### TECHNICAL FIELD

The present technology relates generally to transcription factors for modifying plant metabolism, nucleic acid molecules that encode such transcription factors, and methods of using these nucleic acids to modulate alkaloid production in plants and for producing plant and plant cells having altered alkaloid content.

### BACKGROUND

The following description is provided to assist the understanding of the reader. None of the information provided or references cited are admitted to be prior art.

Pyridine alkaloids play a key role in plant defense mechanisms against herbivore and insect attack as toxic compounds (Sisson and Severson 1990; Facchini, 2001; Voelckel et al., 2001; Kessler and Baldwin, 2002; Kessler et al., 2004; Steppuhn et al., 2004; Dewey and Xie, 2013). In tobacco (Nicotiana tabacum L.) plants, nicotine usually accounts for about 90% of the total alkaloids, with nornicotine, anabasine, and anatabine comprising the majority of the remaining 10% (Saitoh et al., 1985). In the absence of insect herbivory, plants produce only a basal level of nicotine due to the cost of metabolism (Baldwin, 1998). However, this level becomes elevated rapidly in response to wounding (Saunders and Bush, 1979; Baldwin, 1988; Baldwin, 1989). Wound-induced biosynthesis and transportation of jasmonic acid (JA) and its derivatives, such as methyljasmonic acid (MeJA), was identified as a damage signal from shoot to root to promote the biosynthesis of nicotine and other alkaloids (Baldwin, 1989; Baldwin et al., 1994).

Nicotine is exclusively synthesized in the roots of tobacco, subsequently translocated to aerial parts of the plant via xylem, and finally mobilized into the central vacuoles of leaf mesophyll cells mediated by the multidrug and toxic compound extrusion (MATE) transporters (Dawson, 1942; Saunders, 1979; Baldwin 1989; Kitamura et al., 1993; Wink and Roberts, 1998; Morita et al., 2009; Shoji et al., 2009; Shitan et al., 2014). Over the past decades, genes encoding the enzymes in the nicotine biosynthetic pathway have been identified and studied (Bush et al., 1999; Ziegler and Facchini, 2008; Shoji and Hashimoto, 2011; Dewey and Xie, 2013; also FIG. 1). Biochemically, nicotine is formed through the condensation of nicotinic acid (pyridine ring) and N-methyl-Δ¹-pyrrolinium cation (pyrrolidine ring) (Hashimoto and Yamada, 1994). The formation of the pyrrolidine ring starts with the conversion to N-methylputrescine by putrescine N-methyltransferase (PMT) from diamine putrescine, which is synthesized from arginine and ornithine by arginine decarboxylase (ADC) and ornithine decarboxylase (ODC) (Hibi et al., 1992; Imanishi et al., 1998; Riechers and Timko, 1999; Bortolotti et al., 2004; Xu et al., 2004). N-methylputrescine is then oxidized and cyclized to form N-methyl-Δ¹-pyrrolinium cation by N-methylputrescine oxidase (MPO) (Heim et al., 2007; Katoh et al., 2007). The pyridine ring derived from aspartate involves the biosynthesis of nicotinic acid dinucleotide (NAD) controlled by aspartate oxidase (AO), quinolinate synthase (QS) and quinolinic acid phosphoribosyltransferase (QPT) (Sinclair et al., 2000; Katoh et al., 2006; Ryan et al., 2012). The final nicotine ring coupling is mediated by the PIP-family isoflavone reductase-like enzyme (A622) and berberine bridge enzyme-like enzyme (BBL) (DeBoer et al., 2009; Kajikawa et al., 2009; Kajikawa et al., 2011).

The regulation of nicotine biosynthesis involves hormone signal transduction and transcriptional regulation (Dewey and Xie, 2013). Convincing evidence has shown that JA-induced transcriptional upregulation of a suite of genes involved in nicotine biosynthesis is mediated by members from at least two distinct transcription factor families, the AP2 domain-containing ethylene response factor (ERF) family and the MYC2-like basic helix-loop-helix (bHLH) family (De Sutter et al., 2005; Rushton et al., 2008; Shoji et al., 2010; Todd et al., 2010). Two tobacco JA-responsive ERFs, ERF221/ORC1 and ERF10/JAP1, upregulate the gene expression of PMT, one of the key enzymes in nicotine biosynthesis (De Sutter et al., 2005). In 2008, the tobacco AP2/ERF superfamily was studied phylogenetically, and the Group IX ERF members have been identified as main regulators for jasmonate responses in tobacco (Rushton et al., 2008). A cluster of seven Group IX members of the ERF superfamily have been identified as NIC2-locus ERFs which activate the expression of nicotine-related structural genes, such as PMT, ODC, MPO, AO, QS, QPT, A622, and MATE (Shoji et al., 2010; Shoji et al., 2012). Recently, a non-NIC2 locus tobacco ERF, ERF32, has been proven to positively regulate JA-induced nicotine biosynthesis in BY-2 cells (Sears et al., 2014). The transactivation effect of these ERFs is believed to be through the binding to a GCC-box element in the promoter region of several structural genes (Xu and Timko, 2004; Shoji et al., 2010; De Boer et al., 2011; Shoji and Hashimoto, 2012; Shoji and Hashimoto, 2013; Sears et al., 2014).

The specific recognition of the bioactive hormone (+)-7-iso-Jasmonoyl-L-isoleucine (JA-Ile) leads to the degradation of JASMONATE ZIM DOMAIN (JAZ) repressors to release the bHLH family MYC2/3 proteins for transcriptional activation in Arabidopsis (Chini et al., 2007; Thines et al., 2007; Browse, 2009). Recently, JAZ proteins have been manifested as jasmonate co-receptors with the F-box protein CORONATINE INSENSITIVE 1 (COI1), which serves as substrate-recruiting subunit of the Skp1-Cul1-F-box protein (SCF) ubiquitin E3 ligase complex (Sheard et al., 2010; Zhang et al., 2015). In tobacco, in vivo evidence also confirmed the interactions between NtJAZ and NtMYC homologs within the nucleus for the regulation of NtMYC activities in response to JA, as well as the transactivation effects of NtMYC1/2 on a number of structural genes responsible for nicotine biosynthesis through specific binding to the G-box element found in their proximal promoter regions (Xu and Timko, 2004; Shoji et al., 2008; Shoji and Hashimoto, 2011b; Zhang et al., 2012). The suppressed transcript level of NIC2-locus ERF genes in NtMYC2-RNAi tobacco root cells indicated that NtMYC may also directly regulate the transcription of related NtERFs (Shoji and Hashimoto, 2011b; also FIG. 2).

Cumulative study results have demonstrated the functional importance of genes encoding both the structural enzymes and the transcription factors that are involved in nicotine biosynthesis. However, most of these studies focused on the knock-down or repressed effect of gene expression on nicotine or pyridine alkaloid production, and some studies used specified cultured materials, such as root culture and BY-2 cell culture, for genetic transformation (Voelckel et al., 2001; Chintapakorn and Hamill, 2003; Wang et al., 2009; Kajikawa et al., 2009; DeBoer et al., 2009; DeBoer et al., 2011a; Shoji and Hashimoto, 2008; Dalton et al., 2016).

There is a need in the art for methods and compositions for modulating nicotine biosynthesis in plants. The present disclosure satisfies these needs.

### SUMMARY

Disclosed herein are methods and compositions for modulating nicotine biosynthesis in plants.

In one aspect, the present disclosure provides a Nicotiana plant comprising a chimeric nucleic acid construct comprising a nucleotide sequence overexpressing a gene product encoded by NtERF221 operably linked to a heterologous promoter such that NtERF221 is overexpressed relative to a wild-type control plant, whereby the Nicotiana plant accumulates commercial levels of nicotine in its leaves without topping, wherein the nucleotide sequence is selected from the group consisting of: (a) a nucleotide sequence set forth in SEQ ID NO: 1; and (b) a nucleotide sequence that is at least about 90% identical to the nucleotide sequence of (a), and which encodes an NtERF221 transcription factor that positively regulates nicotine biosynthesis.

In some embodiments, the heterologous promoter is selected from the group consisting of a dual CaMV 35S promoter, a Glycine Max Ubiquitin 3 (GmUBI3) gene promoter, and a jasmonate-inducible promoter having the nucleotide sequence set forth in SEQ ID NO: 2. In some embodiments, the heterologous promoter is the jasmonate-inducible promoter having the nucleotide sequence set forth in SEQ ID NO: 2

In some embodiments, the plant is a Nicotiana tabacum plant.

In some embodiments, the present disclosure relates to seeds from the plant, wherein the seeds comprise the chimeric nucleic acid construct.

In some embodiments, the present disclosure relates to a tobacco product comprising the Nicotiana plant, wherein the product has an increased level of nicotine as compared to a tobacco product from a wild-type control plant.

In some embodiments of the plant, the commercial level of nicotine in the tobacco leaves is in the range from about 2.5% to about 6%.

In one aspect, the present disclosure provides a population of tobacco plants characterized by homozygosity for a nucleotide sequence overexpressing a gene product encoded by NtERF221, wherein expression of the gene product is driven by a heterologous promoter such that NtERF221 is overexpressed as compared to a wild-type control tobacco plant, whereby the population stably displays a phenotype comprising a commercial level of nicotine in the tobacco plant leaves without topping, wherein the nucleotide sequence is selected from the group consisting of: (a) a nucleotide sequence set forth in SEQ ID NO: 1; and (b) a nucleotide sequence that is at least about 90% identical to the nucleotide sequence of (a), and which encodes an NtERF221 transcription factor that positively regulates nicotine biosynthesis.

In some embodiments, the commercial level of nicotine in the tobacco leaves is in the range from about 2.5% to about 6%.

In some embodiments, the heterologous promoter is selected from the group consisting of a dual CaMV 35S promoter, a Glycine Max Ubiquitin 3 (GmUBI3) gene promoter, and a jasmonate-inducible promoter having the nucleotide sequence set forth in SEQ ID NO: 2.

In some embodiments, the heterologous promoter is the jasmonate-inducible promoter having the nucleotide sequence set forth in SEQ ID NO: 2

In some embodiments, the plants are Nicotiana tabacum plants.

In some embodiments, the present disclosure relates to seeds from the population of plants, wherein the seeds comprise the chimeric nucleic acid construct.

In some embodiments, the present disclosure relates to a tobacco product comprising the population of tobacco plants, wherein the product has an increased level of nicotine as compared to a tobacco product from wild-type control plants.

In one aspect, the present disclosure provides a method for increasing nicotine in a Nicotiana plant, comprising: (a) introducing into the Nicotiana plant an expression vector comprising a heterologous promoter operably linked to a nucleotide sequence selected from the group consisting of: (i) a nucleotide sequence set forth in SEQ ID NO: 1; and (ii) a nucleotide sequence that is at least about 90% identical to the nucleotide sequence of (i), and which encodes a transcription factor that positively regulates nicotine biosynthesis; and (b) growing the plant under conditions that allow for the expression of a transcription factor that positively regulates nicotine biosynthesis from the nucleotide sequence; wherein expression of the transcription factor results in the plant having an increased nicotine content as compared to a wild-type control plant grown under similar conditions.

In some embodiments, the heterologous promoter is selected from the group consisting of a dual CaMV 35S promoter, a Glycine Max Ubiquitin 3 (GmUBI3) gene promoter, and a jasmonate-inducible promoter having the nucleotide sequence set forth in SEQ ID NO: 2. In some embodiments, the heterologous promoter is the jasmonate-inducible promoter having the nucleotide sequence set forth in SEQ ID NO: 2.

In some embodiments, the method further comprises overexpressing within the Nicotiana plant at least one of NBB1, A622, quinolate phosphoribosyltransferase (QPT), putrescine N-methyltransferase (PMT), ornithine decarboxylase (ODC), aspartate oxidase (AO), quinolinic acid synthase (QS), or N-methylputrescine oxidase (MPO). In some embodiments, the method further comprises overexpressing within the Nicotiana plant at least one additional transcription factor that positively regulates nicotine biosynthesis. In some embodiments, the additional transcription factor that positively regulates nicotinic alkaloid biosynthesis is at least one of NtMYC1a, NtMYC1b, NtMYC2a, or NtMYC2b.

In some embodiments, the vector comprises the nucleotide sequence set forth in SEQ ID NO: 2.

In some embodiments, the method further comprises topping the tobacco plant and/or treating the plant with exogenous jasmonic acid.

### BRIEF DESCRIPTION OF THE DRAWINGS

FIG. 1 is a diagram showing biosynthetic pathways for nicotine and related pyridine alkaloids in tobacco (adapted from Dewey and Xie, 2013). The enzymes or transporters believed to be directly involved in the biosynthesis or accumulation of tobacco alkaloids are in red (i.e., AO, QS, QPT, A622, BBL, ODC, PMT, MPO, NND). Solid arrows, enzymatic reactions defined biochemically; dashed arrows, undefined steps; white arrowheads, spontaneous reactions. A622, a PIP-family oxidoreductase presumably involved in the condensation reactions of a nicotinic acid-derived precursor; ODC, ornithine decarboxylase; ADC, arginine decarboxylase; PMT, putrescine N-methyltransferase; MPO, N-methylputrescine oxidase; AO, aspartate oxidase; QS, quinolinic acid synthase; QPT, qunolinic acid phosphoribosyl transferase; MATE1/2, two homologous multidrug and toxic compound extrusion (MATE)-type transporters implicated in vacuolar sequestration of nicotine in the tobacco roots; SPDS, spermidine synthase; SAMS, S-adenosylmethionine synthase; and SAMDC, S-adenosylmethionine decarboxylase.

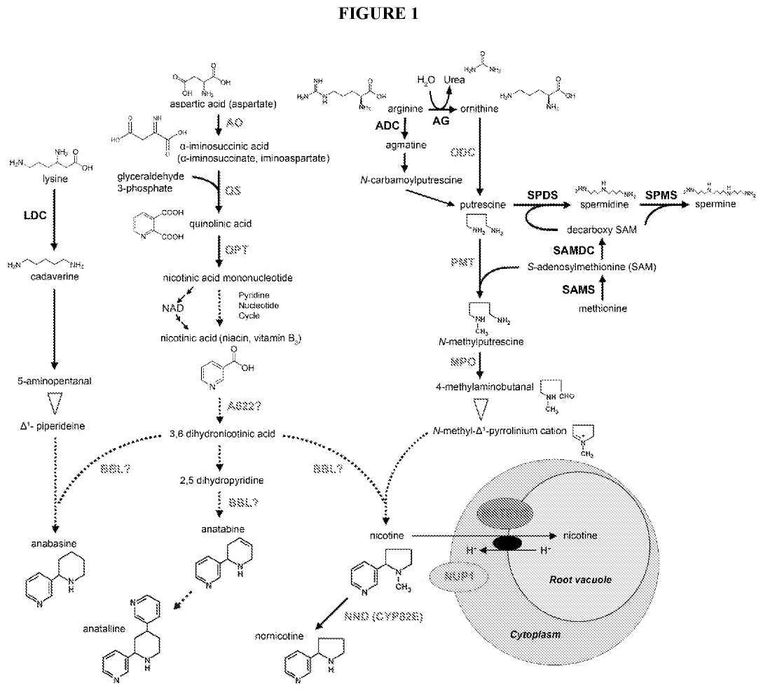

FIG. 2 is a schematic diagram showing a model of JA-mediated transactivation of nicotine biosynthetic genes (adapted from Shoji and Hashimoto, 2011b; Zhang et al., 2012). The presence of JA leads to the formation of JA-Ile, which promotes the interaction between NtJAZ proteins and SCF^{COL1} ubiquitin ligase, leading to the degradation of NtJAZ via the 26S proteasome. This frees NtMYC2 transcription factors to activate the expression of JA-inducible TFs (such as NtERF221) through binding to the G-box-like elements within their promoters, and these TFs then cooperate with NtMYC2 to regulate the transcription of several nicotine biosynthetic genes (such as NtPMT1).

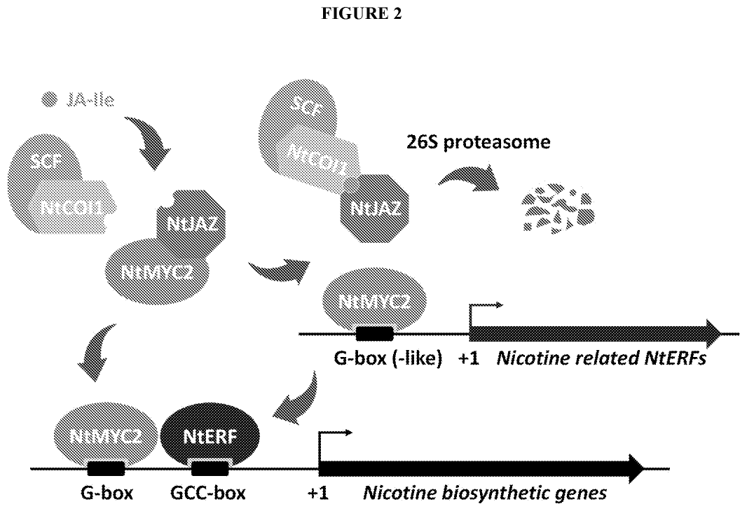

FIGS. 3A-3B are a schematic of vector construction and thin layer chromatography (TLC) analysis of nicotine in wild-type and transgenic tobacco. FIG. 3A: Schematic of the binary vector construction used for overexpression in tobacco. FIG. 3B: TLC assay for the detection of nicotine accumulation in the leaves of 5-week-old wild-type and the T₂ generation NtERF32, NtERF221, or NtMYC2a overexpression lines. The seedlings were treated with 0.1% DMSO (control) or 100 μM MeJA for 48 hours before the leaf tissue was collected for the alkaloid extraction. Arrows indicate nicotine bands, and arrowheads indicate quinaldine as internal control.

FIG. 4 is a series of charts showing the RT-qPCR verification of the transcript levels of NtERF32, NtERF221, and NtMYC2a in wild-type and transgenic tobacco. Two-week-old wild-type and T2 generation NtERF32, NtERF221, and NtMYC2a overexpression seedlings were treated with 0.1% DMSO (control) or 100 μM MeJA for 8 hours before they were collected for RT-qPCR experiment. Relative expression value was normalized to NtEF-1α. Error bars indicate SEM (n=3 PCR replicates). From left to right, for each measurement, 0.1% DMSO is listed first and 100 μM MeJA is listed second.

FIG. 5 is a chart showing the quantification of nicotine in wild-type and transgenic tobacco by GC-MS. Treatment with either 0.1% DMSO (control) or 100 μM MeJA was applied to 5-week-old wild-type or transgenic seedlings for 48 hours. The leaf tissue was collected for alkaloid extraction and GC-MS was performed to quantify nicotine content. For each treatment, six to eight individuals were tested independently for each transgenic line. Statistical analysis was performed with one-way ANOVA and TukeyHSD test for multiple pairwise comparisons. * indicates the level of significance based on the adjusted p-value: ***p<0.001, **p<0.01, * p<0.05.

FIG. 6 is a series of charts showing expression levels of the structural genes that were up-regulated by NtERF221 in wild-type and transgenic tobacco. Two-week-old wild-type or the transgenic tobacco seedlings overexpressing NtERF32, NtERF221, or NtMCY2a were treated with 0.1% DMSO (control) or 100 μM MeJA for 8 hours. Total RNA was collected from at least five individual seedlings for each line. Transcript levels of NtAO, NtODC, NtPMT, NtQPT, and NtQS was measured by RT-qPCR, respectively. Relative expression value was normalized to NtEF-1α. Error bars indicate SEM (n=3 PCR replicates). From left to right, for each measurement, 0.1% DMSO is listed first and 100 μM MeJA is listed second.

### DETAILED DESCRIPTION

### I. Introduction

The present technology relates to the surprising discovery that a stable tobacco plant transformant overexpressing an ERF transcription factor, NtERF221, alone results in a tobacco plant that accumulates commercial levels of nicotine in its leaves without topping. In addition, as Example 1 demonstrates, the present technology relates to the surprising and unexpected finding that the overexpression of this ERF transcription factor alone, bypassing the requirement for MYC and/or MYC plus ERF transcription factor activation, provides a new manner by which nicotine formation can be modulated in tobacco.

To improve leaf quality and production in tobacco, the flowering head and young leaves of the tobacco plant are removed when the first flower of inflorescence appears. This cultivation technique for flue-cured tobacco is known as topping (or decapitating). Tobacco topping activates a comprehensive range of biological processes involving the indole acetic acid (IAA) and jasmonic acid (JA) signaling pathways, and can switch the plant from its reproductive phase to its vegetative phase by altering a number of biological processes in the plant, leading to changes in nicotine biosynthesis and other processes. The JA stimulates the release of MYC transcription factors that can interact with ethylene-responsive element binding factor (ERF) transcription factors to activate the expression of genes responsible for nicotine biosynthesis, thereby stimulating the production of nicotine in the roots and accumulation of nicotine in the leaves of the plant. The increase in nicotine biosynthesis is an important response of tobacco to topping and, without wishing to be bound by theory, to date has been considered to be the only manner by which to achieve substantial nicotine accumulation in tobacco leaves.

The inventors of the present technology examined whether manipulation of transcript levels encoding transcription factors (TFs) previously implicated in the JA-regulated expression of nicotine biosynthetic enzymes could be used as a selective strategy to control nicotine and related alkaloid levels in commercial flue-cured tobacco. As demonstrated herein, the overexpression of specific members of the AP2/ERF family TFs, NtERF32 and NtERF221, and the bHLH family TF, NtMYC2a, alone leads to enhanced nicotine production in flue-cured tobacco and that NtERF221 is particularly effective as a positive regulator of the JA-induced transactivation of a subset of structural genes involved in nicotine biosynthesis, including NtAO, NtODC, NtPMT, NtQPT, and NtQS.

Thus, in some embodiments, the present technology provides a tobacco plant comprising a nucleotide sequence that encodes NtERF221 (ORC1) (e.g., the nucleotide sequence set forth in SEQ ID NO: 1) or biologically active fragments thereof that may be used to genetically manipulate the synthesis of alkaloids (e.g., nicotinic alkaloids) in plants that naturally produce alkaloids. For example, Nicotiana spp. (e.g., N. tabacum, N. rustica, and N. benthamiana) naturally produce nicotinic alkaloids. N. tabacum is an agricultural crop and biotechnological uses of this plant continue to increase. The NtERF221 gene or biologically active fragments thereof may be used in plants or plant cells to increase synthesis of nicotinic alkaloids and related compounds, which may have therapeutic applications.

In some embodiments, the present technology provides a tobacco plant comprising a nucleotide sequence that encodes NtERF221 wherein the expression of the nucleotide sequence is driven by a heterologous promoter such that NtERF221 is overexpressed relative to a wild-type plant, whereby the tobacco plant accumulates commercial levels of nicotine in its leaves without the need for topping. In some embodiments, the heterologous promoter is selected from a dual CaMV 35S promoter, a Glycine Max Ubiquitin 3 (GmUBI3) gene promoter or a novel jasmonate-inducible promoter having a nucleotide sequence as set forth in SEQ ID NO: 2. Thus, in some embodiments, the present technology provides methods for increasing nicotinic alkaloid production in plants and plant cells by genetically engineering overexpression of NtERF221. In some embodiments, the present technology provides methods for increasing nicotine alkaloid production in plants and plant cells by genetically engineering overexpression of NtERF221 and at least one MYC transcription factor gene selected from the group of related NtMYC family members consisting of, but not limited to, NtMYC1a, NtMYC1b, NtMYC2a, and NtMYC2b. The open reading frame (ORF) of the NtMYC1a gene, set forth in SEQ ID NO: 3, encodes the polypeptide sequence set forth in SEQ ID NO: 4. The ORF of the NtMYC1b gene, set forth in SEQ ID NO: 5, encodes the polypeptide sequence set forth in SEQ ID NO: 6. The full-length sequence of the NtMYC2a gene is set forth in SEQ ID NO: 9. The NtMYC2a polypeptide sequence is set forth in SEQ ID NO: 10. The full-length sequence of the NtMYC2b gene is set forth in SEQ ID NO: 9. The NtMYC2b polypeptide sequence is set forth in SEQ ID NO: 10. In some embodiments, the nicotine content of the tobacco plant may be further increased by combining the overexpression of NtERF221 with a technique such as topping or treatment of the plant with exogenous jasmonic acid. In some embodiments, the nicotine content of the tobacco plant may be further increased by combining the overexpression of NtERF221 and at least one MYC transcription factor with a technique such as topping or treatment of the plant with exogenous jasmonic acid.

In some embodiments, a synergistic effect on the production of nicotinic alkaloids is produced by the combined overexpression of NtERF221 and at least one MYC transcription factor gene selected from the group consisting of NtMYC1a, NtMYC1b, NtMYC2a, and NtMYC2b. NtERF221 or biologically active fragments thereof may also be used to genetically engineer suppression of nicotinic alkaloid synthesis to create tobacco varieties containing zero or low nicotine levels for use as low-toxicity production platforms for the production of plant-made pharmaceuticals (e.g., recombinant proteins and antibodies) or as industrial, food, and biomass crops. In some embodiments, a synergistic effect on the production of nicotinic alkaloids is produced by the combination of overexpression of NtERF221 and a technique such as topping or treatment of the plant with exogenous jasmonic acid. In some embodiments, a synergistic effect on the production of nicotinic alkaloids is produced by the combination of overexpression of NtERF221 and at least one MYC transcription factor gene and a technique such as topping or treatment of the plant with exogenous jasmonic acid.

In some embodiments, the commercial level of tobacco leaf nicotine that is achieved without topping is at least about 2.5% to about 6.0% or more. In some embodiments, the commercial level of tobacco leaf nicotine is at least about 3%, at least about 3.5%, at least about 4.0%, at least about 4.5%, at least about 5.0%, at least about 5.1%, at least about 5.2%, at least about 5.3%, at least about 5.4%, at least about 5.5%, at least about 5.6%, at least about 5.7%, at least about 5.8%, at least about 5.9%, or at least about 6.0% or more.

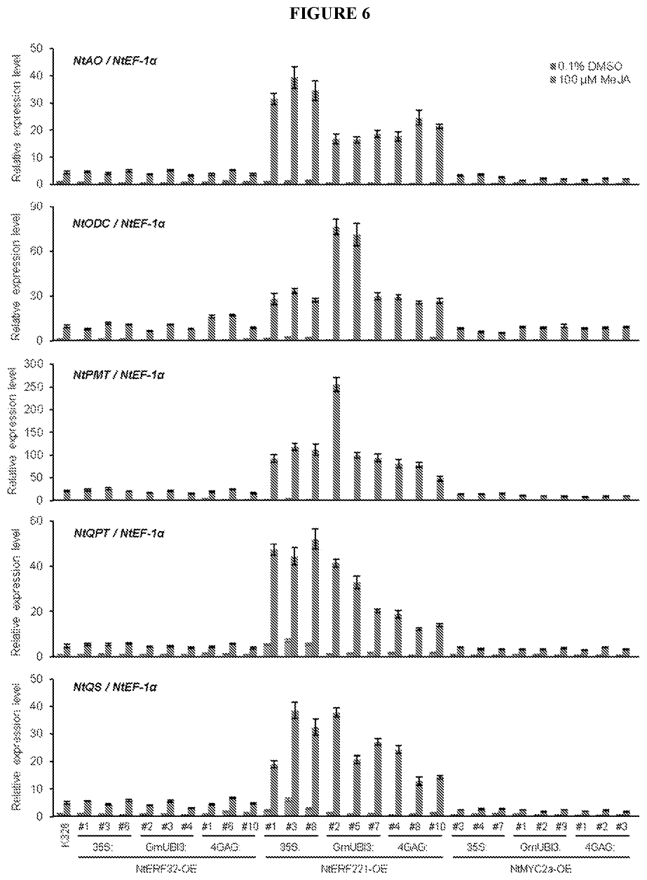

Since the identification of the NIC2-locus ERF genes in tobacco, NtERF189 has been extensively studied for the effect on alkaloid production and stress responses in cultured tobacco roots or cells (Shoji et al., 2010; Shoji and Hashimoto, 2011a,b). The DNA-binding and transcriptional activation properties of NtERF189 have also been well studied (Shoji and Hashimoto, 2012). Phylogenetically, NtERF221 and NtERF189 as well as several other NIC2-locus ERFs are closely related within the same clade/subgroup of the Group IX NtERFs (Sears et al., 2014). NtERF189 has been shown to be able to up-regulate the transcript levels of NtPMT, NtODC, NtMPO, NtAO, NtQS, NtQPT, NtA622 and NtMATE1/2 in transgenic hairy roots (Shoji et al., 2010). Several GCC-box-like sequences were identified to be the binding sites for NtERF189 in the promoters of NtPMT, NtQPT, NtODC and NtMATE (Hashimoto, 2011a; Shoji and Hashimoto, 2012). As described herein, in transgenic tobacco overexpressing NtERF221, the JA-induced transcript accumulation of NtAO, NtODC, NtPMT, NtQPT, and NtQS were greatly up-regulated compared to the wild-type (FIG. 6). This suggests that NtERF221 and NtERF189 may share similar recognition sites in transactivating their target structural genes involved in nicotine biosynthesis.

### II. Modulating Alkaloid Production in Plants

The disclosure of the present technology relates to tobacco plants homozygous for and overexpressing a nucleotide sequence encoding NtERF221, and the use of NtERF221 or biologically active fragments thereof in methods for modulating alkaloid production in plants.

A. Increasing Alkaloid Production

In some embodiments, the present technology relates to increasing alkaloids in plants by overexpressing a transcription factor with a positive regulatory effect on alkaloid production. The NtERF221 gene or its open reading frame (SEQ ID NO: 1) may be used to engineer overproduction of alkaloids, for example, nicotinic alkaloids (e.g., nicotine) in plants or plant cells.

Alkaloids, such as nicotine, can be increased by overexpressing one or more genes encoding enzymes in the alkaloid biosynthesis pathway. See, e.g., Sato et al., Proc. Natl. Acad. Sci. U.S.A. 98(1):367-72 (2001). The effect of overexpressing PMT alone on nicotine content of leaves yields an increase of only 40%, despite 4- to 8-fold increases in PMT transcript levels in roots, suggesting that limitations at other steps of the pathway prevented a larger effect. Accordingly, the present technology contemplates that overexpressing a transcription factor with a positive regulatory effect on alkaloid production (e.g., NtERF221) and at least one at least one alkaloid biosynthesis gene, such as A622, NBB1 (BBL), quinolate phosphoribosyltransferase (QPT), putrescine N-methyltransferase (PMT), ornithine decarboxylase (ODC), aspartate oxidase (AO), quinolinic acid synthase (QS), and/or N-methylputrescine oxidase (MPO), will result in greater alkaloid production than up-regulating the transcription factor or the alkaloid biosynthesis gene alone. Additionally or alternatively, overexpressing more than one additional gene encoding a transcription factor that positively regulates alkaloid production (e.g., a MYC transcription factor such as NtMYC1a, NtMYC1b, NtMYC2a, and/or NtMYC2b) may further increase alkaloids levels in a plant.

Pursuant to this aspect of the present technology, a nucleic acid construct comprising NtERF221, its open reading frame, or a biologically active fragment thereof, and at least one of A622, NBB1, QPT, PMT, ODC, AO, QS, or MPO is introduced into a plant cell. An illustrative nucleic acid construct may comprise, for example, both NtERF221 or a biologically active fragment thereof and QPT. Similarly, for example, a genetically engineered plant overexpressing NtERF221 and QPT may be produced by crossing a transgenic plant overexpressing NtERF221 with a transgenic plant overexpressing QPT. Following successive rounds of crossing and selection, a genetically engineered plant overexpressing NtERF221 and QPT can be selected.

B. Decreasing Alkaloid Production

Alkaloid production may be reduced by suppression of an endogenous gene encoding a transcription factor that positively regulates alkaloid production using the NtERF221 transcription factor gene sequence of the present technology in a number of ways generally known in the art, for example, RNA interference (RNAi) techniques, artificial microRNA techniques, virus-induced gene silencing (VIGS) techniques, antisense techniques, sense co-suppression techniques, and targeted mutagenesis techniques. Accordingly, the present technology provides methodology and constructs for decreasing alkaloid content in a plant by suppressing NtERF221. Suppressing more than one gene encoding a transcription factor that positively regulates alkaloid production (e.g., NtMYC1a, NtMYC1b, NtMYC2a, and/or NtMYC2b) may further decrease alkaloids levels in a plant.

Previous reports indicate that suppressing an alkaloid biosynthesis gene in Nicotiana decreases nicotinic alkaloid content. For example, suppressing QPT reduces nicotine levels. (See, e.g., U.S. Pat. No. 6,586,661). Suppressing A622 or NBB1 also reduces nicotine levels (see, e.g., WO 2006/109197), as does suppressing PMT (see, e.g., Chintapakorn & Hamill, Plant Mol. Biol. 53:87-105 (2003)) or MPO (see, e.g., WO 2008/020333 and WO 2008/008844; Katoh et al., Plant Cell Physiol. 48(3): 550-4 (2007)). Accordingly, the present technology contemplates further decreasing nicotinic alkaloid content by suppressing one or more of A622, NBB1, QPT, PMT, ODC, AO, QS, and MPO, and suppressing NtERF221. Pursuant to this aspect of the present technology, a nucleic acid construct comprising at least a biologically active fragment of NtERF221 and at least a biologically active fragment of one or more of A622, NBB1, QPT, PMT, ODC, AO, QS, and MPO are introduced into a cell or plant. An illustrative nucleic acid construct may comprise both a biologically active fragment of NtERF221 and QPT.

C. Genetic Engineering of Plants and Cells Using Transcription Factor Sequences that Regulate Alkaloid Production

I. Transcription Factor Sequences

Transcription factor genes of the present technology include the sequence set forth in SEQ ID NO: 1 including biologically active fragments thereof of at least about 15 contiguous nucleic acids up to about 680 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, or about 680 contiguous nucleic acids. In some embodiments, transcription factor genes of the present technology include the sequence set forth in SEQ ID NO: 1 including biologically active fragments thereof of at least about 21 consecutive nucleotides, which are of a sufficient length as to be useful in induction of gene silencing in plants (Hamilton & Baulcombe, Science, 286:950-952 (1999)).

The present technology also includes “variants” of SEQ ID NO: 1 with one or more bases deleted, substituted, inserted, or added, which variant codes for a polypeptide that regulates alkaloid biosynthesis activity. Accordingly, sequences having “base sequences with one or more bases deleted, substituted, inserted, or added” retain physiological activity even when the encoded amino acid sequence has one or more amino acids substituted, deleted, inserted, or added. Additionally, multiple forms of NtERF221 may exist, which may be due to post-translational modification of a gene product, or to multiple forms of the transcription factor gene. Nucleotide sequences that have such modifications and that code for an NtERF221 transcription factor that regulates alkaloid biosynthesis are included within the scope of the present technology.

For example, the poly A tail or 5′- or 3′-end, nontranslated regions may be deleted, and bases may be deleted to the extent that amino acids are deleted. Bases may also be substituted, as long as no frame shift results. Bases also may be “added” to the extent that amino acids are added. However, it is essential that any such modification does not result in the loss of transcription factor activity that regulates alkaloid biosynthesis. A modified DNA in this context can be obtained by modifying the DNA base sequences of the present technology so that amino acids at specific sites in the encoded polypeptide are substituted, deleted, inserted, or added by site-specific mutagenesis, for example. (See Zoller & Smith, Nucleic Acid Res. 10:6487-500 (1982)).

A transcription factor sequence can be synthesized ab initio from the appropriate bases, for example, by using an appropriate protein sequence disclosed herein as a guide to create a DNA molecule that, though different from the native DNA sequence, results in the production of a protein with the same or similar amino acid sequence.

Unless otherwise indicated, all nucleotide sequences determined by sequencing a DNA molecule herein were determined using an automated DNA sequencer, such as the Model 3730xl from Applied Biosystems, Inc. Therefore, as is known in the art for any DNA sequence determined by this automated approach, any nucleotide sequence determined herein may contain some errors. Nucleotide sequences determined by automation are typically at least about 95% identical, more typically at least about 96% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence may be completely different from the amino acid sequence actually encoded by the sequenced DNA molecule, beginning at the point of such an insertion or deletion.

For purposes of the present technology, two sequences hybridize under stringent conditions when they form a double-stranded complex in a hybridization solution of 6×SSE, 0.5% SDS, 5×Denhardt's solution and 100 μg of non-specific carrier DNA. See Ausubel, et al., supra, at section 2.9, supplement 27 (1994). Sequences may hybridize at “moderate stringency,” which is defined as a temperature of 60° C. in a hybridization solution of 6×SSE, 0.5% SDS, 5×Denhardt's solution and 100 μg of non-specific carrier DNA. For “high stringency” hybridization, the temperature is increased to 68° C. Following the moderate stringency hybridization reaction, the nucleotides are washed in a solution of 2×SSE plus 0.05% SDS for five times at room temperature, with subsequent washes with 0.1×SSC plus 0.1% SOS at 60° C. for 1 h. For high stringency, the wash temperature is increased to 68° C. For the purpose of the technology, hybridized nucleotides are those that are detected using 1 ng of a radiolabeled probe having a specific radioactivity of 10,000 cpm/ng, where the hybridized nucleotides are clearly visible following exposure to X-ray film at −70° C. for no more than 72 hours.

The present technology encompasses nucleic acid molecules which are at least about 90%, about 91%, about 92%, about 93%, about 94%, about 95%, about 96%, about 97%, about 98%, about 99% or 100% identical to a nucleic acid sequence described in SEQ ID NO: 1. Differences between two nucleic acid sequences may occur at the 5′ or 3′ terminal positions of the reference nucleotide sequence or anywhere between those terminal positions, interspersed either individually among nucleotides in the reference sequence or in one or more contiguous groups within the reference sequence.

II. Nucleic Acid Constructs

In some embodiments of the present technology, a sequence that increases the activity of a transcription factor that regulates alkaloid biosynthesis is incorporated into a nucleic acid construct that is suitable for introducing into a plant or cell. Thus, such a nucleic acid construct can be used to overexpress NtERF221, and optionally at least one of A622, NBB1, QPT, PMT, ODC, AO, QS, MPO, NtMYC1a, NtMYC1b, NtMYC2a, or NtMYC2b in a plant or cell.

Recombinant nucleic acid constructs may be made using standard techniques. For example, the DNA sequence for transcription may be obtained by treating a vector containing the sequence with restriction enzymes to cut out the appropriate segment. The DNA sequence for transcription may also be generated by annealing and ligating synthetic oligonucleotides or by using synthetic oligonucleotides in a polymerase chain reaction (PCR) to give suitable restriction sites at each end. The DNA sequence then is cloned into a vector containing suitable regulatory elements, such as upstream promoter and downstream terminator sequences.

In some embodiments of the present technology, nucleic acid constructs comprise a sequence encoding a transcription factor (i.e., NtERF221) that regulates alkaloid biosynthesis operably linked to one or more regulatory or control sequences, which drive expression of the transcription factor-encoding sequence in certain cell types, organs, or tissues without unduly affecting normal development or physiology.

Promoters useful for expression of a nucleic acid sequence introduced into a cell to either decrease or increase expression of a transcription factor that regulates alkaloid biosynthesis may be constitutive promoters, such as the carnation etched ring virus (CERV), cauliflower mosaic virus (CaMV) 35S promoter, or more particularly the double enhanced cauliflower mosaic virus promoter, comprising two CaMV 35S promoters in tandem (referred to as a “Double 35S” promoter). In some embodiments, the promoter is a Glycine Max Ubiquitin 3 (GmUBI3) gene promoter. Tissue-specific, tissue-preferred, cell type-specific, and inducible promoters may be desirable under certain circumstances. For example, a tissue-specific promoter allows for overexpression in certain tissues without affecting expression in other tissues. In some embodiments, the present technology relates to a novel jasmonate (JA)-inducible promoter in which four copies of the GAG regulatory motif and the minimal promoter originated from NtPMT1a promoter are fused together (4GAG) to give tissue specific and JA-regulated expression consistent with alkaloid formation (SEQ ID NO: 2).

Additional exemplary promoters include promoters which are active in root tissues, such as the tobacco RB7promoter (see, e.g., Hsu et al., Pestic. Sci. 44:9-19 (1995); U.S. Pat. No. 5,459,252), maize promoter CRWAQ81 (see, e.g., U.S. Patent Publication No. 2005/0097633); the Arabidopsis ARSK1 promoter (see, e.g., Hwang & Goodman, Plant J. 8:37-43 (1995)), the maize MR7 promoter (see, e.g., U.S. Pat. No. 5,837,848), the maize ZRP2 promoter (see, e.g., U.S. Pat. No. 5,633,363), the maize MTL promoter (see, e.g., U.S. Pat. Nos. 5,466,785 and 6,018,099) the maize MRS1, MRS2, MRS3, and MRS4 promoters (see, e.g., U.S. Patent Publication No. 2005/0010974), an Arabidopsis cryptic promoter (see, e.g., U.S. Patent Publication No. 2003/0106105) and promoters that are activated under conditions that result in elevated expression of enzymes involved in nicotine biosynthesis such as the tobacco RD2 promoter (see, e.g., U.S. Pat. No. 5,837,876), PMT promoters (see, e.g., Shoji et al., Plant Cell Physiol. 41:831-39 (2000); WO 2002/038588), or an A622 promoter (see, e.g., Shoji et al., Plant Mol. Biol. 50:427-40 (2002)).

The vectors of the present technology may also contain termination sequences, which are positioned downstream of the nucleic acid molecules of the present technology, such that transcription of mRNA is terminated, and polyA sequences added. Exemplary terminators include Agrobacterium tumefaciens nopaline synthase terminator (Tnos), Agrobacterium tumefaciens mannopine synthase terminator (Tmas), and the CaMV 35S terminator (T35S). Termination regions include the pea ribulose bisphosphate carboxylase small subunit termination region (TrbcS) or the Tnos termination region. The expression vector also may contain enhancers, start codons, splicing signal sequences, and targeting sequences.

Expression vectors of the present technology may also contain a selection marker by which transformed cells can be identified in culture. The marker may be associated with the heterologous nucleic acid molecule, i.e., the gene operably linked to a promoter. As used herein, the term “marker” refers to a gene encoding a trait or a phenotype that permits the selection of, or the screening for, a plant or cell containing the marker. In plants, for example, the marker gene will encode antibiotic or herbicide resistance. This allows for selection of transformed cells from among cells that are not transformed or transfected.

Examples of suitable selectable markers include but are not limited to adenosine deaminase, dihydrofolate reductase, hygromycin-B-phosphotransferase, thymidine kinase, xanthine-guanine phospho-ribosyltransferase, glyphosate and glufosinate resistance, and amino-glycoside 3′-O-phosphotransferase (kanamycin, neomycin and G418 resistance). These markers may include resistance to G418, hygromycin, bleomycin, kanamycin, and gentamicin. The construct may also contain the selectable marker gene bar that confers resistance to herbicidal phosphinothricin analogs like ammonium gluphosinate. See, e.g., Thompson et al., EMBO J. 9:2519-23 (1987)). Other suitable selection markers known in the art may also be used.

Visible markers such as green florescent protein (GFP) may be used. Methods for identifying or selecting transformed plants based on the control of cell division have also been described. See, e.g., WO 2000/052168 and WO 2001/059086.

Replication sequences, of bacterial or viral origin, may also be included to allow the vector to be cloned in a bacterial or phage host. Preferably, a broad host range prokaryotic origin of replication is used. A selectable marker for bacteria may be included to allow selection of bacterial cells bearing the desired construct. Suitable prokaryotic selectable markers also include resistance to antibiotics such as kanamycin or tetracycline.

Other nucleic acid sequences encoding additional functions may also be present in the vector, as is known in the art. For example, when Agrobacterium is the host, T-DNA sequences may be included to facilitate the subsequent transfer to and incorporation into plant chromosomes.

Such gene constructs may suitably be screened for activity by transformation into a host plant via Agrobacterium and screening for modified alkaloid levels.

Suitably, the nucleotide sequences for the genes may be extracted from the GenBank™ nucleotide database and searched for restriction enzymes that do not cut. These restriction sites may be added to the genes by conventional methods such as incorporating these sites in PCR primers or by sub-cloning.

Constructs may be comprised within a vector, such as an expression vector adapted for expression in an appropriate host (plant) cell. It will be appreciated that any vector which is capable of producing a plant comprising the introduced DNA sequence will be sufficient.

Suitable vectors are well known to those skilled in the art and are described in general technical references such as Pouwels et al., Cloning Vectors, A Laboratory Manual, Elsevier, Amsterdam (1986). Examples of suitable vectors include the Ti plasmid vectors.

In some embodiments, the present technology provides expression vectors that enable the overexpression of NtERF221, for modulating the production levels of nicotine and other alkaloids, including various flavonoids. In some embodiments, the expression vectors of the present technology further enable the overexpression of at least one of NtMYC1a, NtMYC1b, NtMYC2a, and NtMYC2b. These expression vectors can be transiently introduced into host plant cells or stably integrated into the genomes of host plant cells to generate transgenic plants by various methods known to persons skilled in the art. When these expression vectors are stably integrated into the genomes of host plant cells to generate stable cell lines or transgenic plants, the overexpression of NtERF221 alone or in combination with an alkaloid biosynthesis enzyme or another transcription factor, such as NtMYC1a, NtMYC1b, NtMYC2a, or NtMYC2b, can be deployed as a method for modulating the promoter activation of endogenous promoters that are responsive to this transcription factor. Host plant cells can be further manipulated to receive heterologous promoter constructs that are responsive to NtERF221. Host plant cells can be also be further manipulated to receive heterologous promoter constructs that have been modified by incorporating one or more GAG motifs upstream of the core elements of the heterologous promoter of interest. In some embodiments, the promoter is a jasmonate (JA)-inducible promoter as set forth in SEQ ID NO: 2.

With respect to the expression vectors described below, various genes that encode enzymes involved in biosynthetic pathways for the production of alkaloids, flavonoids, and nicotine can be suitable as transgenes that can be operably linked to a promoter of interest.

In some embodiments, an expression vector comprises a promoter operably linked to the cDNA encoding NtERF221. In another embodiment, a plant cell line comprises an expression vector comprising a promoter operably linked to the cDNA encoding NtERF221. In another embodiment, a transgenic plant comprises an expression vector comprising a promoter operably linked to the cDNA encoding NtERF221. In some embodiments, the transgenic plants are further characterized by homozygosity for and stable expression of NtERF221. In another embodiment, methods for genetically modulating the production of alkaloids, flavonoids, and nicotine are provided, comprising: introducing an expression vector comprising a promoter operably linked to the cDNA encoding NtERF221. In some embodiments, the expression vector further comprises a promoter operably linked to cDNA encoding at least one of NtMYC1a, NtMYC1b, NtMYC2a, and NtMYC2b.

In another embodiment, an expression vector comprises (i) a first promoter operably linked to cDNA encoding NtERF221, and (ii) a second promoter operably linked to cDNA encoding an enzyme involved in the biosynthesis of alkaloids. In another embodiment, a plant cell line comprises (i) an expression vector comprising a first promoter operably linked to cDNA encoding NtERF221, and (ii) a second promoter operably linked to cDNA encoding an enzyme involved in the biosynthesis of alkaloids. In another embodiment, a transgenic plant comprises (i) an expression vector comprising a first promoter operably linked to cDNA encoding NtERF221, and (ii) a second promoter operably linked to cDNA encoding an enzyme involved in the biosynthesis of alkaloids. In another embodiment, methods for genetically modulating the production level of alkaloids are provided, comprising introducing an expression vector comprising (a) a first promoter operably linked to cDNA encoding NtERF221, and (b) a second promoter operably linked to cDNA encoding an enzyme involved in the biosynthesis of alkaloids. In some embodiments, the expression vector further comprises a promoter operably linked to cDNA encoding at least one of NtMYC1a, NtMYC1b, NtMYC2a, and NtMYC2b. In some embodiments, the enzyme involved in alkaloid biosynthesis comprises one or more of A622, NBB1, QPT, PMT, ODC, AO, QS, or MPO.

In another embodiment, an expression vector comprises (i) a first promoter operably linked to cDNA encoding NtERF221, (ii) and a second promoter operably linked to cDNA encoding an enzyme involved in the biosynthesis of flavonoids. In another embodiment, a plant cell line comprises (i) an expression vector comprising a first promoter operably linked to cDNA encoding NtERF221, and (ii) a second promoter operably linked to cDNA encoding an enzyme involved in the biosynthesis of flavonoids. In another embodiment, a transgenic plant comprises an expression vector comprising (i) a first promoter operably linked to cDNA encoding NtERF221, and (ii) a second promoter operably linked to cDNA encoding an enzyme involved in the biosynthesis of flavonoids. In some embodiments, the expression vector further comprises a promoter operably linked to cDNA encoding at least one of NtMYC1a, NtMYC1b, NtMYC2a, and NtMYC2b. In another embodiment, methods for modulating the production level of flavonoids are provided, comprising introducing an expression vector comprising (i) a first promoter operably linked to cDNA encoding NtERF221, and (ii) a second promoter operably linked to cDNA encoding an enzyme involved in the biosynthesis of flavonoids. In some embodiments of the methods, the expression vector further comprises a promoter operably linked to cDNA encoding at least one of NtMYC1a, NtMYC1b, NtMYC2a, and NtMYC2b.

In another embodiment, an expression vector comprises (i) a first promoter operably linked to cDNA encoding NtERF221, and (ii) a second promoter operably linked to cDNA encoding an enzyme involved in nicotine biosynthesis. In another embodiment, a plant cell line comprises an expression vector comprising (i) a first promoter operably linked to cDNA encoding NtERF221, and (ii) a second promoter operably linked to cDNA encoding an enzyme involved in nicotine biosynthesis. In another embodiment, a transgenic plant comprises an expression vector comprising (i) a first promoter operably linked to cDNA encoding NtERF221, and (ii) a second promoter operably linked to cDNA encoding an enzyme involved in nicotine biosynthesis. In some embodiments, the expression vector further comprises a promoter operably linked to cDNA encoding at least one of NtMYC1a, NtMYC1b, NtMYC2a, and NtMYC2b. In some embodiments, the enzyme involved in nicotine biosynthesis is one or more of A622, NBB1, QPT, PMT, ODC, AO, QS, or MPO. In some embodiments, the enzyme involved in nicotine biosynthesis is PMT. In another embodiment, methods for genetically modulating the production level of nicotine are provided, comprising introducing an expression vector comprising (i) a first promoter operably linked to cDNA encoding NtERF221, and (ii) a second promoter operably linked to cDNA encoding an enzyme involved in nicotine biosynthesis. In some embodiments of the methods, the expression vector further comprises a promoter operably linked to cDNA encoding at least one of NtMYC1a, NtMYC1b, NtMYC2a, and NtMYC2b.

Another embodiment is directed to an isolated cDNA encoding NtERF221 (SEQ ID NO: 1), or biologically active fragments thereof. Another embodiment is directed to an isolated cDNA encoding NtERF221 and having at least about 90%, about 91%, about 92%, about 93%, about 94%, about 95%, about 96%, about 97%, about 98%, or about 99% sequence identity to SEQ ID NO: 1, or biologically active variant fragments thereof.

Another embodiment is directed to an expression vector comprising a first sequence comprising an isolated cDNA encoding NtERF221 and having at least about 90%, about 91%, about 92%, about 93%, about 94%, about 95%, about 96%, about 97%, about 98%, or about 99% sequence identity to SEQ ID NO: 1, or biologically active fragments thereof. In some embodiments, the expression vector further comprises an additional sequence comprising an isolated cDNA encoding at least one of NtMYC1a, NtMYC1b, NtMYC2a, and NtMYC2b, and having at least about 85%, about 90%, about 91%, about 92%, about 93%, about 94%, about 95%, about 96%, about 97%, about 98%, about 99% or 100% sequence identity to SEQ ID NOs: 3, 5, 7, and 9, respectively, or fragments thereof.

Another embodiment is directed to a plant cell line comprising an expression vector comprising an isolated cDNA encoding NtERF221 and having at least about 90%, about 91%, about 92%, about 93%, about 94%, about 95%, about 96%, about 97%, about 98%, or about 99% sequence identity to SEQ ID NO: 1, or fragments thereof. In some embodiments, the expression vector further comprises an additional sequence comprising an isolated cDNA encoding at least one of NtMYC1a, NtMYC1b, NtMYC2a, and NtMYC2b, and having at least about 85%, about 90%, about 91%, about 92%, about 93%, about 94%, about 95%, about 96%, about 97%, about 98%, about 99% or 100% sequence identity to SEQ ID NOs: 3, 5, 7, and 9, respectively, or fragments thereof.

Another embodiment is directed to a transgenic plant comprising an expression vector comprising an isolated cDNA encoding NtERF221 and having at least about 90%, about 91%, about 92%, about 93%, about 94%, about 95%, about 96%, about 97%, about 98%, or about 99% sequence identity to SEQ ID NO: 1, or biologically active fragments thereof. In some embodiments, the expression vector further comprises a second sequence comprising an isolated cDNA encoding at least one of NtMYC1a, NtMYC1b, NtMYC2a, and NtMYC2b, and having at least about 85%, about 90%, about 91%, about 92%, about 93%, about 94%, about 95%, about 96%, about 97%, about 98%, about 99% or 100% sequence identity to SEQ ID NOs: 3, 5, 7, and 9, respectively, or fragments thereof.

Another embodiment is directed to a method for genetically regulating nicotine levels in plants, comprising introducing into a plant an expression vector comprising an isolated cDNA encoding NtERF221 and having at least about 90%, about 91%, about 92%, about 93%, about 94%, about 95%, about 96%, about 97%, about 98%, or about 99% sequence identity to SEQ ID NO: 1, or fragments thereof. In some embodiments, the expression vector further comprises a second sequence comprising an isolated cDNA encoding at least one of NtMYC1a, NtMYC1b, NtMYC2a, and NtMYC2b, and having at least about 85%, about 90%, about 91%, about 92%, about 93%, about 94%, about 95%, about 96%, about 97%, about 98%, about 99% or 100% sequence identity to SEQ ID NOs: 3, 5, 7, and 9, respectively, or fragments thereof.

III. Methodology for Suppressing a Transcription Factor that Regulates Alkaloid Production

In some embodiments of the present technology, methods and constructs are provided for suppressing a transcription factor that regulates alkaloid production, altering alkaloid levels, and producing plants with altered alkaloid levels. Examples of methods that may be used for suppressing a transcription factor that regulates alkaloid production (e.g., NtERF221) include antisense, sense co-suppression, RNAi, artificial microRNA, virus-induced gene silencing (VIGS), antisense, sense co-suppression, and targeted mutagenesis approaches.

RNAi techniques involve stable transformation using RNAi plasmid constructs (Helliwell & Waterhouse, Methods Enzymol. 392:24-35 (2005)). Such plasmids are composed of a fragment of the target gene to be silenced in an inverted repeat structure. The inverted repeats are separated by a spacer, often an intron. The RNAi construct driven by a suitable promoter, for example, the Cauliflower mosaic virus (CaMV) 35S promoter, is integrated into the plant genome and subsequent transcription of the transgene leads to an RNA molecule that folds back on itself to form a double-stranded hairpin RNA. This double-stranded RNA structure is recognized by the plant and cut into small RNAs (about 21 nucleotides long) called small interfering RNAs (siRNAs). siRNAs associate with a protein complex (RISC) which goes on to direct degradation of the mRNA for the target gene.

Artificial microRNA (amiRNA) techniques exploit the microRNA (miRNA) pathway that functions to silence endogenous genes in plants and other eukaryotes (Schwab et al., Plant Cell 18:1121-33 (2006); Alvarez et al., Plant Cell 18:1134-51 (2006)). In this method, 21-nucleotide-long fragments of the gene to be silenced are introduced into a pre-miRNA gene to form a pre-amiRNA construct. The pre-miRNA construct is transferred into the plant genome using transformation methods apparent to one skilled in the art. After transcription of the pre-amiRNA, processing yields amiRNAs that target genes, which share nucleotide identity with the 21 nucleotide amiRNA sequence.

In RNAi silencing techniques, two factors can influence the choice of length of the fragment. The shorter the fragment the less frequently effective silencing will be achieved, but very long hairpins increase the chance of recombination in bacterial host strains. The effectiveness of silencing also appears to be gene dependent and could reflect accessibility of target mRNA or the relative abundances of the target mRNA and the hpRNA in cells in which the gene is active. A fragment length of between 100 and 800 bp, preferably between 300 and 600 bp, is generally suitable to maximize the efficiency of silencing obtained. The other consideration is the part of the gene to be targeted. 5′ UTR, coding region, and 3′ UTR fragments can be used with equally good results. As the mechanism of silencing depends on sequence homology there is potential for cross-silencing of related mRNA sequences. Where this is not desirable, a region with low sequence similarity to other sequences, such as a 5′ or 3′ UTR, should be chosen. The rule for avoiding cross-homology silencing appears to be to use sequences that do not have blocks of sequence identity of over 20 bases between the construct and the non-target gene sequences. Many of these same principles apply to selection of target regions for designing amiRNAs.

Virus-induced gene silencing (VIGS) techniques are a variation of RNAi techniques that exploits the endogenous-antiviral defenses of plants. Infection of plants with recombinant VIGS viruses containing fragments of host DNA leads to post-transcriptional gene silencing for the target gene. In one embodiment, a tobacco rattle virus (TRV) based VIGS system can be used. Tobacco rattle virus based VIGS systems are described for example, in Baulcombe, Curr. Opin. Plant Biol. 2:109-113 (1999); Lu et al., Methods 30:296-303 (2003); Ratcliff et al., The Plant Journal 25:237-245 (2001); and U.S. Pat. No. 7,229,829.

Antisense techniques involve introducing into a plant an antisense oligonucleotide that will bind to the messenger RNA (mRNA) produced by the gene of interest. The “antisense” oligonucleotide has a base sequence complementary to the gene's messenger RNA (mRNA), which is called the “sense” sequence. Activity of the sense segment of the mRNA is blocked by the anti-sense mRNA segment, thereby effectively inactivating gene expression. Application of antisense to gene silencing in plants is described in more detail in Stam et al., Plant J. 21 27-42 (2000).

Sense co-suppression techniques involve introducing a highly expressed sense transgene into a plant resulting in reduced expression of both the transgene and the endogenous gene (Depicker and van Montagu, Curr. Opin. Cell Biol. 9: 373-82 (1997)). The effect depends on sequence identity between transgene and endogenous gene.

Targeted mutagenesis techniques, for example TILLING (Targeting Induced Local Lesions IN Genomes) and “delete-a-gene” using fast-neutron bombardment, may be used to knockout gene function in a plant (Henikoff et al., Plant Physiol. 135: 630-6 (2004); Li et al., Plant J. 27: 235-242 (2001)). TILLING involves treating seeds or individual cells with a mutagen to cause point mutations that are then discovered in genes of interest using a sensitive method for single-nucleotide mutation detection. Detection of desired mutations (e.g., mutations resulting in the inactivation of the gene product of interest) may be accomplished, for example, by PCR methods. For example, oligonucleotide primers derived from the gene of interest may be prepared and PCR may be used to amplify regions of the gene of interest from plants in the mutagenized population. Amplified mutant genes may be annealed to wild-type genes to find mismatches between the mutant genes and wild-type genes. Detected differences may be traced back to the plants which had the mutant gene thereby revealing which mutagenized plants will have the desired expression (e.g. silencing of the gene of interest). These plants may then be selectively bred to produce a population having the desired expression. TILLING can provide an allelic series that includes missense and knockout mutations, which exhibit reduced expression of the targeted gene. TILLING is touted as a possible approach to gene knockout that does not involve introduction of transgenes, and therefore may be more acceptable to consumers. Fast-neutron bombardment induces mutations, i.e., deletions, in plant genomes that can also be detected using PCR in a manner similar to TILLING.

IV. Host Plants and Cells

In some embodiments, the present technology relates to the genetic manipulation of a plant or cell via introducing a polynucleotide sequence that encodes a transcription factor that regulates alkaloid biosynthesis (e.g., NtERF221). Accordingly, the present technology provides methodology and constructs for reducing or increasing alkaloid synthesis in a plant. Additionally, the present technology provides methods for producing alkaloids and related compounds in a plant cell.

The plants utilized in the present technology may include the class of alkaloid-producing higher plants amenable to genetic engineering techniques, including both monocotyledonous and dicotyledonous plants, as well as gymnosperms. In some embodiments, the alkaloid-producing plant includes a nicotinic alkaloid-producing plant of the Nicotiana, Duboisia, Solanum, Anthocercis, and Salpiglossis genera in the Solanaceae or the Eclipta and Zinnia genera in the Compositae.

As known in the art, there are a number of ways by which genes and gene constructs can be introduced into plants, and a combination of plant transformation and tissue culture techniques have been successfully integrated into effective strategies for creating transgenic crop plants.

These methods, which can be used in the present technology, have been described elsewhere (Potrykus, Annu. Rev. Plant Physiol. Plant Mol. Biol. 42:205-225 (1991); Vasil, Plant Mol. Biol. 5:925-937 (1994); Walden and Wingender, Trends Biotechnol. 13:324-331 (1995); Songstad et al., Plant Cell, Tissue and Organ Culture 40:1-15 (1995)), and are well known to persons skilled in the art. For example, one skilled in the art will certainly be aware that, in addition to Agrobacterium-mediated transformation of Arabidopsis by vacuum infiltration (Bechtold et al., C.R. Acad. Sci. Ser. III Sci. Vie, 316:1194-1199 (1993)) or wound inoculation (Katavic et al., Mol. Gen. Genet. 245:363-370 (1994)), it is equally possible to transform other plant and crop species, using Agrobacterium Ti-plasmid-mediated transformation (e.g., hypocotyl (DeBlock et al., Plant Physiol. 91:694-701 (1989)) or cotyledonary petiole (Moloney et al., Plant Cell Rep. 8:238-242 (1989) wound infection), particle bombardment/biolistic methods (Sanford et al., J. Part. Sci. Technol. 5:27-37 (1987); Nehra et al., Plant J. 5: 285-297 (1994); Becker et al., Plant J. 5: 299-307 (1994)), or polyethylene glycol-assisted protoplast transformation (Rhodes et al., Science 240: 204-207 (1988); Shimamoto et al., Nature 335: 274-276 (1989)) methods.

Agrobacterium rhizogenes may be used to produce transgenic hairy roots cultures of plants, including tobacco, as described, for example, by Guillon et al., Curr. Opin. Plant Biol. 9:341-6 (2006). “Tobacco hairy roots” refers to tobacco roots that have T-DNA from an Ri plasmid of Agrobacterium rhizogenes integrated in the genome and grow in culture without supplementation of auxin and other phytohormones. Tobacco hairy roots produce nicotinic alkaloids as roots of a whole tobacco plant do.

Additionally, plants may be transformed by Rhizobium, Sinorhizobium, or Mesorhizobium transformation. (Broothaerts et al., Nature 433:629-633 (2005)).

After transformation of the plant cells or plant, those plant cells or plants into which the desired DNA has been incorporated may be assessed for zygosity and selected by such methods as antibiotic resistance, herbicide resistance, tolerance to amino-acid analogues or using phenotypic markers (See, e.g., Passricha et al., J. Biol. Methods 3(3):e45 (2016)).

Various assays may be used to determine whether the plant cell shows a change in gene expression, for example, Northern blotting or quantitative reverse transcriptase PCR (RT-PCR). Whole transgenic plants may be regenerated from the transformed cell by conventional methods. Such transgenic plants may be propagated and self-pollinated to produce homozygous lines. Such plants produce seeds containing the genes for the introduced trait and can be grown to produce plants that will produce the selected phenotype.

Modified alkaloid content, effected in accordance with the present technology, can be combined with other traits of interest, such as disease resistance, pest resistance, high yield or other traits. For example, a stable genetically engineered transformant that contains a suitable transgene that modifies alkaloid content may be employed to introgress a modified alkaloid content trait into a desirable commercially acceptable genetic background, thereby obtaining a cultivar or variety that combines a modified alkaloid level with said desirable background. For example, a genetically engineered tobacco plant with reduced nicotine may be employed to introgress the reduced nicotine trait into a tobacco cultivar with disease resistance trait, such as resistance to TMV, blank shank, or blue mold. Alternatively, cells of a modified alkaloid content plant of the present technology may be transformed with nucleic acid constructs conferring other traits of interest.

The present technology also contemplates genetically engineering a cell with a nucleic acid sequence encoding a transcription factor that regulates alkaloid biosynthesis (e.g., NtERF221).

Additionally, cells expressing alkaloid biosynthesis genes may be supplied with precursors to increase substrate availability for alkaloid synthesis. Cells may be supplied with analogs of precursors which may be incorporated into analogs of naturally occurring alkaloids.

Constructs according to the present technology may be introduced into any plant cell, using a suitable technique, such as Agrobacterium-mediated transformation, particle bombardment, electroporation, and polyethylene glycol fusion, or cationic lipid-mediated transfection.

Such cells may be genetically engineered with a nucleic acid construct of the present technology without the use of a selectable or visible marker and transgenic organisms may be identified by detecting the presence of the introduced construct. The presence of a protein, polypeptide, or nucleic acid molecule in a particular cell can be measured to determine if, for example, a cell has been successfully transformed or transfected. For example, and as routine in the art, the presence of the introduced construct can be detected by PCR or other suitable methods for detecting a specific nucleic acid or polypeptide sequence. Additionally, genetically engineered cells may be identified by recognizing differences in the growth rate or a morphological feature of a transformed cell compared to the growth rate or a morphological feature of a non-transformed cell that is cultured under similar conditions. See WO 2004/076625.

The present technology also contemplates transgenic plant cell cultures comprising genetically engineered plant cells transformed with the nucleic acid molecules described herein and expressing NtERF221. The cells may also express at least one additional transcription factor gene such as NtMYC1a, NtMYC1b, NtMYC2a, or NtMYC2b, and/or at least one nicotine biosynthesis gene such as A622, NBB1, QPT, PMT, ODC, AO, QS, or MPO

The present technology also contemplates cell culture systems comprising genetically engineered cells transformed with the nucleic acid molecules described herein and expressing NtERF221. It has been shown that transgenic hairy root cultures overexpressing PMT provide an effective means for large-scale commercial production of scopolamine, a pharmaceutically important tropane alkaloid. Zhang et al., Proc. Nat'l Acad. Sci. USA 101:6786-91 (2004). Accordingly, large-scale or commercial quantities of nicotinic alkaloids can be produced in tobacco hairy root culture by overexpressing NtERF221. Likewise, the present technology contemplates cell culture systems, such as bacterial or insect cell cultures, for producing large-scale or commercial quantities of nicotinic alkaloids, nicotine analogs, or nicotine precursors by expressing NtERF221. The cells may also express at least one additional transcription factor gene such as NtMYC1a, NtMYC1b, NtMYC2a, or NtMYC2b, and/or at least one nicotine biosynthesis gene such as A622, NBB1, QPT, PMT, ODC, AO, QS, or MPO.

D. Quantifying Alkaloid Content

In some embodiments of the present technology, genetically engineered plants and cells are characterized by reduced alkaloid content.

A quantitative reduction in alkaloid levels can be assayed by several methods, as for example by quantification based on gas-liquid chromatography, high performance liquid chromatography, radio-immunoassays, and enzyme-linked immunosorbent assays.

In describing a plant of the present technology, the phrase “decreased alkaloid plant” or “reduced alkaloid plant” encompasses a plant that has a decrease in alkaloid content to a level less than about 50%, about 40%, about 30%, about 25%, about 20%, about 15%, about 10%, about 9%, about 8%, about 7%, about 6%, about 5%, about 4%, about 3%, about 2% or about 1% of the alkaloid content of a control plant of the same species or variety.

In some embodiments of the present technology, genetically engineered plants are characterized by increased alkaloid content. Similarly, genetically engineered cells are characterized by increased alkaloid production.

In describing a plant of the present technology, the phrase “increased alkaloid plant” encompasses a genetically engineered plant that has an increase in alkaloid content greater than about 10%, about 25%, about 30%, about 40%, about 50%, about 75%, about 100%, about 125%, about 150%, about 175%, or about 200% of the alkaloid content of a control plant of the same species or variety.

A successfully genetically engineered cell is characterized by increased alkaloid synthesis. For example, a genetically engineered cell of the present technology may produce more nicotine compared to a control cell.

A quantitative increase in nicotinic alkaloid levels can be assayed by several methods, as for example by quantification based on gas-liquid chromatography, high performance liquid chromatography, radio-immunoassays, and enzyme-linked immunosorbent assays.

### III. Products

The polynucleotide sequences that encode the NtERF221 transcription factor that regulates alkaloid biosynthesis may be used for production of plants with altered alkaloid levels. Such plants may have useful properties, such as increased pest resistance in the case of increased-alkaloid plants, or reduced toxicity and increased palatability in the case of decreased-alkaloid plants.

Plants of the present technology may be useful in the production of products derived from harvested portions of the plants. For example, decreased-alkaloid tobacco plants may be useful in the production of reduced-nicotine cigarettes for smoking cessation. Increased-alkaloid tobacco plants may be useful in the production of modified risk tobacco products.

Additionally, plants and cells of the present technology may be useful in the production of alkaloids or alkaloid analogs including nicotine analogs, which may be used as therapeutics, insecticides, or synthetic intermediates. To this end, large-scale or commercial quantities of alkaloids and related compounds can be produced by a variety of methods, including extracting compounds from genetically engineered plant, cell, or culture system, including but not limited to hairy root cultures, suspension cultures, callus cultures, and shoot cultures.

### IV. Definitions

All technical terms employed in this specification are commonly used in biochemistry, molecular biology and agriculture; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al., (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al., (John Wiley & Sons, Inc., 2002); Genome Analysis: A Laboratory Manual, vol. 1-2, ed. Green et al., (Cold Spring Harbor Laboratory Press, Cold Spring Harbor, N.Y., 1997). Methodology involving plant biology techniques are described here and also are described in detail in treatises such as Methods In Plant Molecular Biology: A Laboratory Course Manual, ed. Maliga et al., (Cold Spring Harbor Laboratory Press, Cold Spring Harbor, N.Y., 1995).

An “alkaloid” is a nitrogen-containing basic compound found in plants and produced by secondary metabolism. A “pyrrolidine alkaloid” is an alkaloid containing a pyrrolidine ring as part of its molecular structure, for example, nicotine. Nicotine and related alkaloids are also referred to as pyridine alkaloids in the published literature. A “pyridine alkaloid” is an alkaloid containing a pyridine ring as part of its molecular structure, for example, nicotine. A “nicotinic alkaloid” is nicotine or an alkaloid that is structurally related to nicotine and that is synthesized from a compound produced in the nicotine biosynthesis pathway. Illustrative nicotinic alkaloids include but are not limited to nicotine, nornicotine, anatabine, anabasine, anatalline, N-methylanatabine, N-methylanabasine, myosmine, anabaseine, formylnornicotine, nicotyrine, and cotinine. Other very minor nicotinic alkaloids in tobacco leaf are reported, for example, in Hecht et al., Accounts of Chemical Research 12: 92-98 (1979); Tso, T. G., Production, Physiology and Biochemistry of Tobacco Plant. Ideals Inc., Beltsville, Mo. (1990).

As used herein “alkaloid content” means the total amount of alkaloids found in a plant, for example, in terms of pg/g dry weight (DW) or ng/mg fresh weight (FW). “Nicotine content” means the total amount of nicotine found in a plant, for example, in terms of mg/g DW or FW.

A “chimeric nucleic acid” comprises a coding sequence or fragment thereof linked to a nucleotide sequence that is different from the nucleotide sequence with which it is associated in cells in which the coding sequence occurs naturally.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein.

“Endogenous nucleic acid” or “endogenous sequence” is “native” to, i.e., indigenous to, the plant or organism that is to be genetically engineered. It refers to a nucleic acid, gene, polynucleotide, DNA, RNA, mRNA, or cDNA molecule that is present in the genome of a plant or organism that is to be genetically engineered.

“Exogenous nucleic acid” refers to a nucleic acid, DNA or RNA, which has been introduced into a cell (or the cell's ancestor) through the efforts of humans. Such exogenous nucleic acid may be a copy of a sequence which is naturally found in the cell into which it was introduced, or fragments thereof.

As used herein, “expression” denotes the production of an RNA product through transcription of a gene or the production of the polypeptide product encoded by a nucleotide sequence. “Overexpression” or “up-regulation” is used to indicate that expression of a particular gene sequence or variant thereof, in a cell or plant, including all progeny plants derived thereof, has been increased by genetic engineering, relative to a control cell or plant (e.g., “NtERF221 overexpression”).

“Genetic engineering” encompasses any methodology for introducing a nucleic acid or specific mutation into a host organism. For example, a plant is genetically engineered when it is transformed with a polynucleotide sequence that suppresses expression of a gene, such that expression of a target gene is reduced compared to a control plant. A plant is genetically engineered when a polynucleotide sequence is introduced that results in the expression of a novel gene in the plant, or an increase in the level of a gene product that is naturally found in the plants. In the present context, “genetically engineered” includes transgenic plants and plant cells, as well as plants and plant cells produced by means of targeted mutagenesis effected, for example, through the use of chimeric RNA/DNA oligonucleotides, as described by Beetham et al., Proc. Natl. Acad. Sci. U.S.A. 96: 8774-8778 (1999) and Zhu et al., Proc. Natl. Acad. Sci. U.S.A. 96: 8768-8773 (1999), or so-called “recombinagenic olionucleobases,” as described in International patent publication WO 2003/013226. Likewise, a genetically engineered plant or plant cell may be produced by the introduction of a modified virus, which, in turn, causes a genetic modification in the host, with results similar to those produced in a transgenic plant. See, e.g., U.S. Pat. No. 4,407,956. Additionally, a genetically engineered plant or plant cell may be the product of any native approach (i.e., involving no foreign nucleotide sequences), implemented by introducing only nucleic acid sequences derived from the host plant species or from a sexually compatible plant species. See, e.g., U.S. Patent Application No. 2004/0107455.

“Heterologous nucleic acid” refers to a nucleic acid, DNA, or RNA, which has been introduced into a cell (or the cell's ancestor), and which is not a copy of a sequence naturally found in the cell into which it is introduced. Such heterologous nucleic acid may comprise segments that are a copy of a sequence that is naturally found in the cell into which it has been introduced, or fragments thereof.

“Homozygous” and “homozygosity” may be used interchangeably herein. A plant is homozygous when the alleles of a gene residing on a homologous chromosome pair are identical. All gametes arising from this plant are identical at that gene locus and such plants do not segregate on selfing. Thus, non-segregating genotypes constitute homozygous populations.

By “isolated nucleic acid molecule” is intended a nucleic acid molecule, DNA, or RNA, which has been removed from its native environment. For example, recombinant DNA molecules contained in a DNA construct are considered isolated for the purposes of the present technology. Further examples of isolated DNA molecules include recombinant DNA molecules maintained in heterologous host cells or DNA molecules that are purified, partially or substantially, in solution. Isolated RNA molecules include in vitro RNA transcripts of the DNA molecules of the present technology. Isolated nucleic acid molecules, according to the present technology, further include such molecules produced synthetically.

“Plant” is a term that encompasses whole plants, plant organs (e.g., leaves, stems, roots, etc.), seeds, differentiated or undifferentiated plant cells, and progeny of the same. Plant material includes without limitation seeds, suspension cultures, embryos, meristematic regions, callus tissues, leaves, roots, shoots, stems, fruit, gametophytes, sporophytes, pollen, and microspores.

“Plant cell culture” means cultures of plant units such as, for example, protoplasts, cell culture cells, cells in plant tissues, pollen, pollen tubes, ovules, embryo sacs, zygotes, and embryos at various stages of development. In some embodiments of the present technology, a transgenic tissue culture or transgenic plant cell culture is provided, wherein the transgenic tissue or cell culture comprises a nucleic acid molecule of the present technology.

“Decreased alkaloid plant” or “reduced alkaloid plant” encompasses a genetically engineered plant that has a decrease in alkaloid content to a level less than 50%, and preferably less than 10%, 5%, or 1% of the alkaloid content of a control plant of the same species or variety.

“Increased alkaloid plant” encompasses a genetically engineered plant that has an increase in alkaloid content greater than 10%, and preferably greater than 50%, 100%, or 200% of the alkaloid content of a control plant of the same species or variety.

“Promoter” connotes a region of DNA upstream from the start of transcription that is involved in recognition and binding of RNA polymerase and other proteins to initiate transcription. A “constitutive promoter” is one that is active throughout the life of the plant and under most environmental conditions. Tissue-specific, tissue-preferred, cell type-specific, and inducible promoters constitute the class of “non-constitutive promoters.” “Operably linked” refers to a functional linkage between a promoter and a second sequence, where the promoter sequence initiates and mediates transcription of the DNA sequence corresponding to the second sequence. In general, “operably linked” means that the nucleic acid sequences being linked are contiguous.

“Sequence identity” or “identity” in the context of two polynucleotide (nucleic acid) or polypeptide sequences includes reference to the residues in the two sequences that are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties, such as charge and hydrophobicity, and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers & Miller, Computer Applic. Biol. Sci. 4: 11-17 (1988), as implemented in the program PC/GENE (Intelligenetics, Mountain View, Calif., USA).

Use in this description of a percentage of sequence identity denotes a value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The terms “suppression” or “down-regulation” are used synonymously to indicate that expression of a particular gene sequence variant thereof, in a cell or plant, including all progeny plants derived thereof, has been reduced by genetic engineering, relative to a control cell or plant (e.g., “NtERF221 down-regulation”).

As used herein, a “synergistic effect” refers to a greater-than-additive effect which is produced by a combination of at least two compounds (e.g., the effect produced by a combined overexpression of at least two transcription factors, such as NtERF221 and at least one MYC transcription factor gene preferably selected from the group of related NtMYC family members consisting of, but not limited to, NtMYC1a, NtMYC1b, NtMYC2a, and NtMYC2b and or an ERF transcription such as NtERF241) or techniques (e.g., the effect produced by the combination of overexpression of one or more transcription factors, such as NtERF221, and topping or exogenous jasmonic acid treatment of the tobacco plant), and which exceeds that which would otherwise result from the individual compound (e.g., the effect produced by the overexpression of a single transcription factor, such as NtERF221 alone) or technique.

“Tobacco” or “tobacco plant” refers to any species in the Nicotiana genus that produces nicotinic alkaloids, including but not limited to the following: Nicotiana acaulis, Nicotiana acuminata, Nicotiana acuminata var. multzjlora, Nicotiana africana, Nicotiana alata, Nicotiana amplexicaulis, Nicotiana arentsii, Nicotiana attenuata, Nicotiana benavidesii, Nicotiana benthamiana, Nicotiana bigelovii, Nicotiana bonariensis, Nicotiana cavicola, Nicotiana clevelandii, Nicotiana cordifolia, Nicotiana corymbosa, Nicotiana debneyi, Nicotiana excelsior, Nicotiana forgetiana, Nicotiana fragrans, Nicotiana glauca, Nicotiana glutinosa, Nicotiana goodspeedii, Nicotiana gossei, Nicotiana hybrid, Nicotiana ingulba, Nicotiana kawakamii, Nicotiana knightiana, Nicotiana langsdorfi, Nicotiana linearis, Nicotiana longiflora, Nicotiana maritima, Nicotiana megalosiphon, Nicotiana miersii, Nicotiana noctiflora, Nicotiana nudicaulis, Nicotiana obtusifolia, Nicotiana occidentalis, Nicotiana occidentalis subsp. hesperis, Nicotiana otophora, Nicotiana paniculata, Nicotiana pauczjlora, Nicotiana petunioides, Nicotiana plumbaginifolia, Nicotiana quadrivalvis, Nicotiana raimondii, Nicotiana repanda, Nicotiana rosulata, Nicotiana rosulata subsp. ingulba, Nicotiana rotundifolia, Nicotiana rustica, Nicotiana setchellii, Nicotiana simulans, Nicotiana solanifolia, Nicotiana spegauinii, Nicotiana stocktonii, Nicotiana suaveolens, Nicotiana sylvestris, Nicotiana tabacum, Nicotiana thyrsiflora, Nicotiana tomentosa, Nicotiana tomentosifomis, Nicotiana trigonophylla, Nicotiana umbratica, Nicotiana undulata, Nicotiana velutina, Nicotiana wigandioides, and interspecific hybrids of the above.

“Tobacco product” refers to a product comprising material produced by a Nicotiana plant, including for example, cut tobacco, shredded tobacco, nicotine gum and patches for smoking cessation, cigarette tobacco including expanded (puffed) and reconstituted tobacco, cigar tobacco, pipe tobacco, cigarettes, cigars, and all forms of smokeless tobacco such as chewing tobacco, snuff, snus, and lozenges.

A “transcription factor” is a protein that binds that binds to DNA regions, typically promoter regions, using DNA binding domains and increases or decreases the transcription of specific genes. A transcription factor “positively regulates” alkaloid biosynthesis if expression of the transcription factor increases the transcription of one or more genes encoding alkaloid biosynthesis enzymes and increases alkaloid production. A transcription factor “negatively regulates” alkaloid biosynthesis if expression of the transcription factor decreases the transcription of one or more genes encoding alkaloid biosynthesis enzymes and decreases alkaloid production. Transcription factors are classified based on the similarity of their DNA binding domains. (See, e.g., Stegmaier et al., Genome Inform. 15 (2): 276-86 ((2004)). Classes of plant transcription factors include ERF transcription factors; Myc basic helix-loop-helix transcription factors; homeodomain leucine zipper transcription factors; AP2 ethylene-response factor transcription factors; and B3 domain, auxin response factor transcription factors.

A “variant” is a nucleotide or amino acid sequence that deviates from the standard, or given, nucleotide or amino acid sequence of a particular gene or polypeptide. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence, may be considered a variant sequence. A polypeptide variant may have “conservative” changes, wherein a substituted amino acid has similar structural or chemical properties, e.g., replacement of leucine with isoleucine. A polypeptide variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, Md.) software. Variant may also refer to a “shuffled gene” such as those described in Maxygen-assigned patents (see, e.g., U.S. Pat. No. 6,602,986).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The term “biologically active fragment” means a fragment of NtERF221 which can, for example, bind to an antibody that will also bind the full length NtERF221. The term “biologically active fragment” can also mean a fragment of NtERF221 which can, for example, be useful in induction of gene silencing in plants. In some embodiments, a biologically active fragment of NtERF221 can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% of the open reading frame sequence (either amino acid or nucleic acid). SEQ ID NO. 1 depicts the ORF of NtERF221, including the coding region and its 5′ and 3′ upstream and downstream regulatory sequences. SEQ ID NO. 1 is 684 base pairs in length. In some embodiments, a biologically active nucleic acid fragment of NtERF221 can be, for example, at least about 15 contiguous nucleic acids. In yet other embodiments, the biologically active nucleic acid fragment of NtERF221 can be about 15 contiguous nucleic acids up to about 680 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, or about 675 contiguous nucleic acids.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Materials and Methods

Plant materials and transformation. Tobacco plants (Nicotiana tabacum L. var. K326) were used for all the tests and genetic transformations described herein. Wild-type or transgenic seeds were germinated on Murashige and Skoog (MS) plates. For quantitative RT-PCR, germinated small seedlings were transferred onto larger MS plates and grown vertically until two weeks old before treated with 0.1% DMSO (control) or 100 μM MeJA. For TLC and GC-MS analysis, germinated seedlings were transferred in soil and grown under normal condition till five weeks old before DMSO or MeJA treatment. Agrobacterium tumefaciens strain LBA4404 harboring each transgenic vector was used for genetic transformation of tobacco following the experiment procedure described previously (Horsch et al., 1985). At least eight T₀ transgenic plants were confirmed by genomic DNA PCR and then self-pollinated to produce T₁ generation. T₁ plants were screened on MS plates containing 50 mg/L hygromycin, verified by genomic DNA PCR, and then grown in the greenhouse and self-pollinated to produce T₂ generation. The non-segregating T₂ tobacco seedlings were used in this study.

Selection of Non-Segregating T₂ Transgenic Tobacco Seedlings. First, eight to ten T₀ transgenic plants were confirmed by genomic DNA PCR for the existence of the transgenic fragment. Then, they were grown in the greenhouse and self-pollinated to produce the T₁ generation seeds, which were subsequently screened on germination media containing the selective antibiotic, hygromycin (50 mg/L). Germinated seedlings were further verified by genomic DNA PCR. These T₁ plants were then grown in the greenhouse and self-pollinated to produce the T₂ generation seeds. Different T₂ generation seed lots were tested on the same germination media containing hygromycin for segregation evaluation. The non-segregating T₂ transgenic tobacco seedlings were used in this study.

DNA cloning and vector construction. Coding regions (CDS) of NtERF10 (CQ808845), NtERF32 (AB828154), NtERF121 (AY655738), NtERF221 (CQ808982), and NtMYC2a (HM466974) were amplified by polymerase chain reaction (PCR) with Phusion High-Fidelity DNA Polymerase (New England Biolabs) and introduced into Gateway pDONR221 vector via BP recombination reaction for sequence verification. The PCR amplified promoter sequence of Glycine max Ubiquitin-3 (GmUBI3) gene and artificially synthesized 4GAG promoter derived from Nicotiana tabacum PMT1a (NtPMT1a) gene (SEQ ID NO: 2) were used to replace the original dual cauliflower mosaic virus (CaMV) 35S promoter (2× 35S) in the binary vector pMDC32 respectively, namely pGmUBI3-MDC and p4GAG-MDC, for Gateway compatibility. Sequence verified genes in pDONR221 were then sub-cloned into pMDC32, pGmUBI3-MDC and p4GAG-MDC respectively. The resulting constructs were designated as 35S:ERF10, 35S:ERF32, 35S:ERF121, 35S:ERF221, 35S:MYC2a, GmUBI3:ERF10, GmUBI3:ERF32, GmUBI3:ERF121, GmUBI3:ERF221, GmUBI3:MYC2a, 4GAG:ERF10, 4GAG:ERF32, 4GAG:ERF121, 4GAG:ERF221, and 4GAG:MYC2a.

RNA isolation and quantitative reverse transcription-PCR (RT-qPCR). For gene expression analysis, five to six two-week-old seedlings were collected together as one sample for RNA isolation. Total RNA was isolated with TRIzol reagent (Thermo Scientific) following the manufacturer's instructions. DNA contaminants were removed from total RNA with DNase I (RNase-free, New England Biolabs). The DNA-eliminated total RNA was then reverse-transcribed using QuantiTect reverse transcription kit (Qiagen). Quantitative PCR was conducted iTaq™ Universal SYBR® Green supermix (Bio-Rad Laboratories) on an CFX96™ Real-Time PCR detection system (Bio-Rad Laboratories). The relative expression level of each gene was normalized to N. tabacum Elongation Factor 1-alpha (NtEF-1α, Schmidt and Delaney, 2010).

Alkaloid extraction and thin layer chromatography (TLC). Alkaloid extraction was performed as described by Goossens et al., (2003). Briefly, leaves from five-week-old wild-type or transgenic seedlings 48 h after DMSO or MeJA treatment were collected and lyophilized. 10 mg lyophilized tissue were homogenized in liquid nitrogen and basified with 10% NH₄OH. 100 quinaldine was added as an internal standard. Total alkaloids were extracted with CH₂Cl₂, vacuum concentrated and resuspended into 200 μL CH₂Cl₂. For the TLC assay, alkaloid extracts from three individuals of each line were mixed together and then equal amounts of the extracts from different lines were loaded onto a silica gel TLC plate (UV254, Analtech). Separation was done with the mobile phase composed of dichloromethane:methanol:10% NH₄OH (125:15:2). Spots were visualized by the spray with Dragendorff reagent (Sigma-Aldrich).

Gas chromatography-mass spectrometry (GC-MS). For GC-MS analysis of nicotine, alkaloids were extracted with naphthalene-d8 as an internal standard. For each transgenic line, total alkaloid was extracted from six to eight five-week-old independent individuals. Nicotine concentration was measured on a Shimadzu GCMS QP2010 plus system with a protocol developed previously (Goossens et al., (2003); Zhang et al., (2012)). Statistical test was performed by the Analysis of Variance (ANOVA) followed by Tukey's Honest Significant Difference (TukeyHSD) test using R (version 3.4.4).

### Example 1: Increased Nicotine Production by the Overexpression of NtERF221, NtERF32, and NtMYC2a

To clarify respective impact of the genes closely related to nicotine biosynthesis on the actual nicotine accumulation in commercial grade tobacco plants, genes were cloned and overexpressed under the control of different promoters in the flue-cured tobacco variety K326. These genes included five transcription factor (TF) genes previously implicated in controlling nicotine biosynthesis, i.e., NtERF10, NtERF32, NtERF121, NtERF221 and NtMYC2a (Table 1).

TABLE 1
Transcription Factors Modulating Nicotine Biosynthesis

| | | Enzyme or | |
|---|---|---|---|
| Transcription Factor | ID | Transporter | ID |
| NtERF10 (JAP1) | gb\|CQ808845 | NtPMT1a | gb\|AF126810 |
| NtERF32 (ERF2) | gb\|AB828154 | NtQPT2 | gb\|AB038494 |
| NtERF121 (ERF5) | gb\|AY655738 | NtMPO2 | gb\|AB289457 |
| NtERF221 (0RC1) | gb\|CQ808982 | NtMATE1 | gb\|AB286961 |
| NtMYC2a | gb\|HM466974 | NtA622 | gb\|D28505 |

The constructs employed three different promoters: (a) a double enhanced CaMV 35S promoter (2× 35S) to give well defined high level constitutive expression, (b) a constitutive GmUBI3 gene promoter previously shown to be highly expressed in tobacco (Hernandez-Garcia et al., 2010), and (c) a novel jasmonate (JA)-inducible promoter in which four copies of the GAG regulatory motif and the minimal promoter originated from NtPMT1a promoter are fused together (4GAG) to give tissue specific and JA-regulated expression consistent with alkaloid formation (SEQ ID NO: 2).

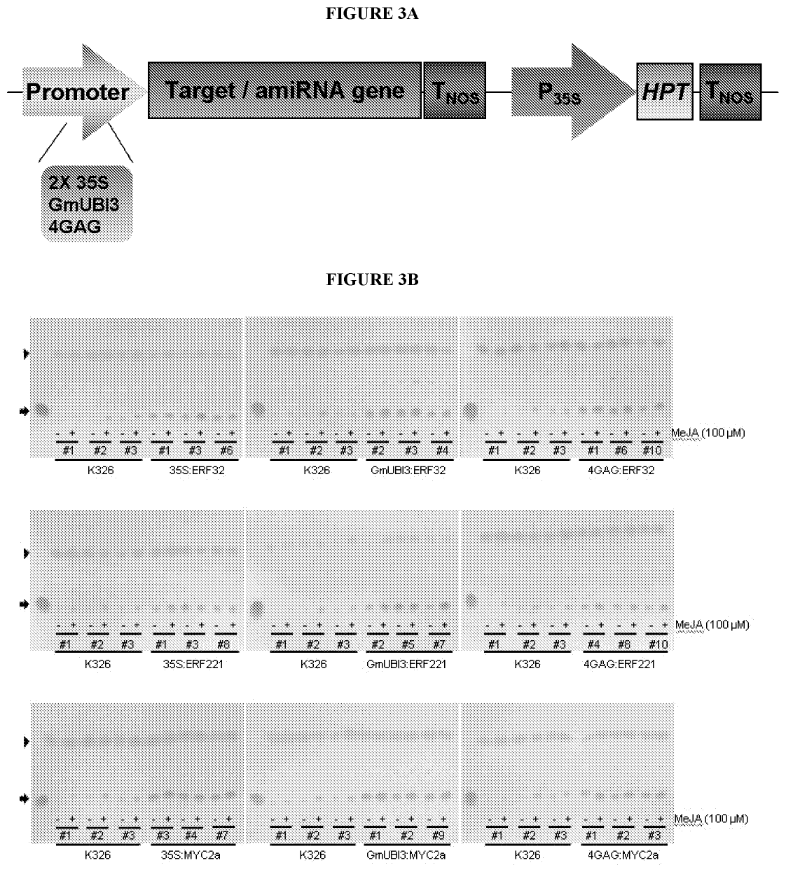

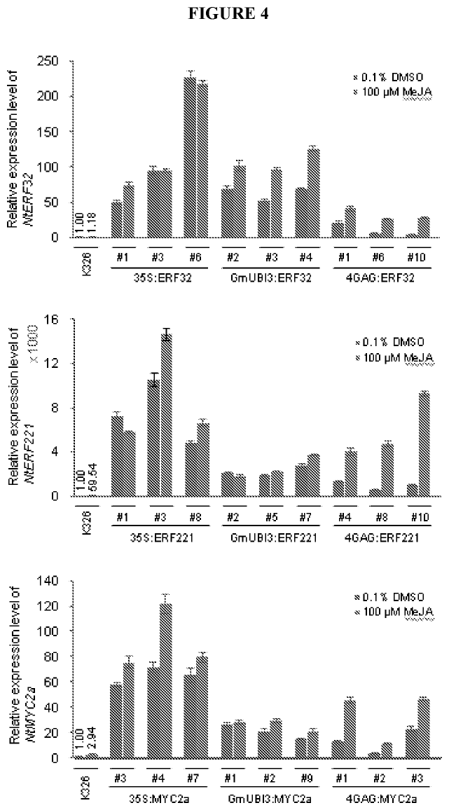

Each gene was put into three different expression constructs respectively under the control of three different promoters described above (FIG. 3A). Transformants were generated by Agrobacterium-mediated transformation with the flue-cured tobacco N. tabacum K326. For each construct, at least eight lines were confirmed to have the transgene stably integrated into the genome by genomic PCR, the intact structure of the transgene was validated and relative level of transgene expression measured by RT-PCR using gene specific primers as is standard practice. The level of nicotine was assessed by TLC analysis to quickly identify the transformants with higher nicotine accumulation than the wild-type. Individuals selected on the basis of elevated nicotine expression phenotype were grown in the greenhouse and self-pollinated manually to advance them from the T₀ to T₁ generations and the resulting T₁ plants were screened for non-segregating progenies. T₁ lines were similarly self-pollinated and the resulting T₂ plants analyzed. TLC results from the T₂ transgenic plants revealed that the overexpression of the NtERF32, NtERF221, and NtMYC2a transgenes resulted in a significantly elevated nicotine accumulation as a consequence of constitutive or conditional transgene overexpression (FIG. 3B). The transcript level of each gene in the T₂ transgenic lines was also compared with that in the wild-type through RT-qPCR. As shown in FIG. 4, constitutive overexpression of NtERF32, NtERF221, or NtMYC2a persisted with its high transcript level when compared to the wild-types with or without MeJA stimulation. When controlled by the 4GAG promoter, these three genes exhibited induced expression pattern by MeJA, although the basal transcript level of each gene were also higher than that in the wild-type (FIG. 4), suggesting that the 4GAG promoter may be very sensitive to the basal/natural level of intracellular JA.

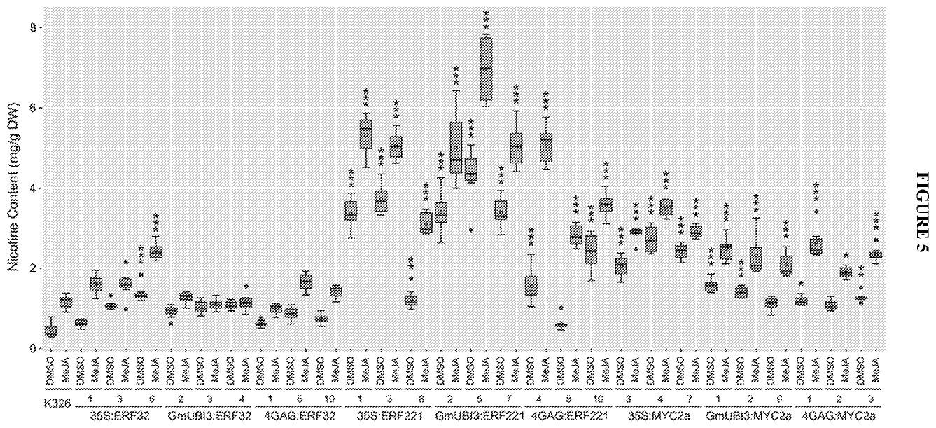

To further quantify nicotine concentration for the comparison between transgenic lines and the wild-types, GC-MS analysis was applied with total alkaloids extracted from the leaf tissue of five-week-old wild-type or transgenic plants treated with DMSO (control) or MeJA. Among different transgenic lines, overexpression of NtERF221 gave the highest nicotine concentration in the leaf (FIG. 5). In comparison with the wild-type, line #5 of the GmUBI3:ERF221 gave roughly 4.5 times higher nicotine concentration without MeJA elicitation and almost 9-fold higher nicotine concentration when treated with MeJA. Approximately 1.5 to 3 fold increase of nicotine accumulation was observed in the transgenic lines overexpressing NtMYC2a compared to the wild-type (FIG. 5). The 35S:MYC2a lines had a little higher nicotine concentration than the GmUBI3:MYC2a and 4GAG:MYC2a lines did, yet it is not as high as that in most of the NtERF221 overexpression lines. Compared to the JA-inducible 4GAG promoter, both constitutive promoters gave higher nicotine production averagely after MeJA treatment (FIG. 5).

Accordingly, these results demonstrate that transgenic tobacco plants overexpressing NtERF32, NtERF221 or NtMYC2a transgenes significantly elevated nicotine accumulation as a consequence of constitutive or conditional transgene overexpression in the plant.

### Example 2: Regulatory Control of the Genes Involved in Nicotine Biosynthesis by NtERF221

Studies on the expression levels of nicotine biosynthetic genes in transgenic materials that overexpress NtERF32, NtERF221, or NtMYC2a provided clues to better understand the relationships and dynamics between TFs and nicotine biosynthesis enzymes. As shown in FIG. 6, the JA-induced transcript accumulation of NtAO, NtODC, NtPMT, NtQPT, and NtQS were far greater in the transgenic tobacco overexpressing NtERF221 than in the wild-type or the other transgenic tobacco tested. Although the expression of these five structural genes were all responsive to MeJA treatment, no obvious difference in the MeJA-induced transcript up-regulation of these five genes could be observed between the wild-type and the NtERF32 or NtMYC2a transgenic tobacco (FIG. 6).

### REFERENCES

Bush, L., Hempfling, W. P., Burton, H. (1999) Biosynthesis of nicotine and related compounds. In: Gorrod, J. W., Jacob, P., III (Eds.), Analytical Determination of Nicotine and Related Compounds and Their Metabolites. Elsevier Science, Amsterdam, pp. 13-45.
Baldwin I T (1988) Damage-induced alkaloids in tobacco: Pot-bound plants are not inducible. J Chem Ecol 14:1113-1120.
Baldwin I T (1989) Mechanism of damaged-induced alkaloid production in wild tobacco. J Chem Ecol 15:1661-1680.
Baldwin I T, Schmelz E A, Ohnmeiss T E (1994) Wound-induced changes in root and shoot jasmonic acid pools correlate with induced nicotine synthesis in Nicotiana sylvestris Spegazzini and Comes. J Chem Ecl 20:2139-2157.
Baldwin, I. T., Zhang, Z. P., Diab, N., Ohnmeiss, T. E., McCloud, E. S., Lynds, G. Y. and Schmelz, E. A. (1997) Quantification, correlations, and manipulations of wound induced changes in jasmonic acid and nicotine in Nicotiana sylvestris. Planta 201: 397-404.
Baldwin I T (1998) Jasmonate-induced responses are costly but benefit plants under attack in native populations. Proc Natl Acad Sci USA 95:8113-8118.
Baldwin, I T (1999) Inducible nicotine production in native Nicotiana as an example of phenotypic plasticity. J. Chem. Ecol. 25: 3-30.
Baldwin, I. T. and Prestin, C. A. (1999) The eco-physiological complexity of plant responses to insect herbivores. Planta 208: 137-145.
Browse, J. (2009) Jasmonate Passes Muster: A Receptor and Targets for the Defense Hormone Annu. Rev. Plant Biol. 60:183-205.
Cane, K. A., Mayer, M., Lidgett, A. J., Michael, A. J., Hamill, J. D. (2005) Molecular analysis of alkaloid metabolism in AABB v. aabb genotype Nicotiana tabacum in response to wounding of aerial tissues and methyl jasmonate treatment of cultured roots. Funct. Plant Biol. 32, 305-320.
Chini A., Fonseca S., Fernandez G., et al., (2007) The JAZ family of repressors is the missing link in jasmonate signaling. Nature: 448, pages 666-671.
Chintapakorn, Y., Hamill, J. D. (2003) Antisense-mediated down-regulation of putrescine N-methyltransferase activity in transgenic Nicotiana tabacum L. can lead to elevated levels of anatabine at the expense of nicotine. Plant Mol. Biol. 53, 87-105.
Dalton H L, Blomstedt C K, Neale A D, Gleadow R, DeBoer K D, Hamill J D (2016) Effects of down-regulating ornithine decarboxylase upon putrescine-associated metabolism and growth in Nicotiana tabacum L. Journal of Experimental Botany, 67(11): 3367-3381.
DeBoer, K. D., Lye, J. C., Aitken, C. D., Su, A. K.-K., Hamill, J. D. (2009) The A622 gene in Nicotiana glauca (tree tobacco): evidence for a functional role in pyridine alkaloid synthesis. Plant Mol. Biol. 69, 299-312.
DeBoer, K. D., Dalton, H. L., Edward, F. J., Hamill, J. D. (2011a) RNAi-mediated downregulation of ornithine decarboxylase (ODC) leads to reduced nicotine and increased anatabine levels in transgenic Nicotiana tabacum L. Phytochemistry 72, 344-355.
DeBoer, K. D., Tilleman, S., Pauwels, L., Bossche, R. V., De Sutter, V., Vanderhaeghen, R., Hilson, P., Hamill, J. D., Goossens, A. (2011b) APETATA2/ETHYLENE RESPONSE FACTOR and basic helix-loop-helix tobacco transcription factors cooperatively mediate jasmonate-elicited nicotine biosynthesis. Plant J. 66, 1053-1065.
Geyter N D, Gholami A, Goormachtig S and Goossens A (2012) Transcriptional machineries in jasmonate-elicited plant secondary metabolism. Trends in Plant Science June, Vol. 17, No. 6.
Hashimoto, T. and Yamada, Y. (1994) Alkaloid biogenesis: molecular aspects. Annu. Rev. Plant Physiol. Plant Mol. Biol. 45: 257-285.
Hibi N, Higashiguchi S, Hashimoto T, Yamada Y (1994) Gene expression in tobacco low-nicotine mutants. Plant Cell 6:723-735.
Horsch, R. B. et al., (1985) A simple and general method for transferring genes into plants. Science 227, 1229-1231.
Imanishi, S., Hashizume, K., Nakakita, M., Kojima, H., Matsubayashi, Y., Hashimoto, T., Sakagami, Y., Yamada, Y. and Nakamura, K. (1998) Differential induction by methyl jasmonate of genes encoding ornithine decarboxylase and other enzymes involved in nicotine biosynthesis in tobacco cell cultures. Plant Mol. Biol. 38: 1101-1111.
Jorg Ziegler and Peter J. Facchini (2008) Alkaloid Biosynthesis: Metabolism and Trafficking. Annu. Rev. Plant Biol. 2008. 59:735-69.
Kajikawa, M., Hirai, N., Hashimoto, T. (2009) A PIP-family protein is required for biosynthesis of tobacco alkaloids. Plant Mol. Biol. 69, 287-298.
Katsir L, Hoo Sun Chung, Abraham J K Koo and Gregg A Howe (2008) Jasmonate signaling: a conserved mechanism of hormone sensing. Current Opinion in Plant Biology 2008, 11:428-435.
Kidd S, Melillo A, Lu R-H, Reed D, Kuno N, Uchida K, Furuya M, Jelesko J (2006) The A and B loci in tobacco regulate a network of stress response genes, few of which are associated with nicotine biosynthesis. Plant Mol Biol 60:699-716.
Legg P D, Collins G B (1971) Inheritance of percent total alkaloids in Nicotiana tabacum L. II. Genetic effects of two loci in Burley 21 9 LA Burley 21 populations. Can J Genet Cytol 13:287-291.
Li F, Wang W, Zhao N, Xiao B, Cao P, et al., (2015) Regulation of Nicotine Biosynthesis by an Endogenous Target Mimicry of MicroRNA in Tobacco. Plant Physiol. 169(2): 1062-1071.
Morita M, Shitan N, Sawada K, Van Montague M C E, Inze D, et al., (2009) Vacuolar transport of nicotine is mediated by a multidrug and toxic compound extrusion (MATE) transporter in Nicotiana tabacum. Proc. Natl. Acad. Sci. USA 106:2447-52.
Nakano T, Nishiuchi T, Suzuki K, Fujimura T, Shinshi H (2006) Studies on transcriptional regulation of endogenous genes by ERF2 transcription factor in tobacco cells. Plant Cell Physiol 47:554-558.
Ohme-Takagi M, Shinshi H (1995) Ethylene-inducible DNA-binding proteins that interact with an ethylene-responsive element. Plant Cell 7:173-182.
Ohnmeiss T. E., McCloud E. S., Lynds G. Y. and Baldwin, I. T. (1997) Within-plant relationships among wounding, jasmonic acid, and nicotine: implications for defense in Nicotiana sylvestris. New Phytologist 137: 441-452.
Sato, F., Hashimoto, T., Hachiya, A., Tamura, K., Choi, K., Morishige, T., Fujimoto, H., Yamada, Y. (2001) Metabolic engineering of plant alkaloid biosynthesis. Proc. Natl. Acad. Sci. USA 98, 367-372.
Saunders J W, Bush L P (1979) Nicotine biosynthetic enzyme activities in Nicotiana tabacum L. genotypes with different alkaloid levels. Plant Physiol 64:236-240.
Shitan N, Minami S, Morita M, Hayashida M, Ito S, Takanashi K, Omote H, Moriyama Y, Sugiyama A, Goossens A, Moriyasu M, Yazaki K (2014) Involvement of the leaf-specific multidrug and toxic compound extrusion (MATE) transporter Nt-JAT2 in vacuolar sequestration of nicotine in Nicotiana tabacum. PLoS One. 9(9): e108789.
Shoji, T., Yamada, Y. and Hashimoto, T. (2000) Jasmonate induction of putrescine N-methyltransferase genes in the root of Nicotiana sylvestris. Plant Cell Physiol. 41: 831-839.
Shoji, T., Winz, R., Iwase, T., Yamada, Y., Hashimoto, T. (2002) Expression patterns of two tobacco isoflavone reductase-like genes and their possible roles in secondary metabolism in tobacco. Plant Mol. Biol. 50, 427-440.
Shoji T, Inai K, Yazaki Y, Sato Y, Takase H, Shitan N, Yazaki K, Goto Y, Toyooka K, Matsuoka K, Hashimoto T (2009) Multidrug and toxic compound extrusion-type transporters implicated in vacuolar sequestration of nicotine in tobacco roots. Plant Physiol. 149(2):708-18.
Shoji T, Kajikawa M, Hashimoto T (2010) Clustered transcription factor genes regulate nicotine biosynthesis in tobacco. Plant Cell 22:3390-3409.
Shoji, T., Hashimoto, T. (2011a) Recruitment of a duplicated primary metabolism gene into the nicotine biosynthesis regulon in tobacco. Plant J. 67, 949-959.
Shoji, T., Hashimoto, T. (2011b) Tobacco MYC2 regulates jasmonate-inducible nicotine biosynthesis genes directly and by way of the NIC2-locus ERF genes. Plant Cell Physiol. 52, 1117-1130.
Shoji T, Hashimoto T (2012) DNA-binding and transcriptional activation properties of tobacco NIC2-locus ERF189 and related transcription factors. Plant Biotechnol 29: 35-42.
Shoji T, Mishima M, Hashimoto T (2013) Divergent DNA-binding specificities of a group of ETHYLENE RESPONSE FACTOR transcription factors involved in plant defense. Plant Physiology 162 (2):977-90.
Shoji T, Hashimoto T (2014) Stress-induced expression of NICOTINE2-locus genes and their homologs encoding Ethylene Response Factor transcription factors in tobacco. Phytochemistry http://dx.doi.org/10.1016/j.phytochem.2014.05.017.
Sinclair, S. J., Murphy, K. J., Birch, C. D. and Hamil, J. D. (2000) Molecular characterization of quinolinate phosphoribosyltransferase (QPRTase) in Nicotiana. Plant Mol. Biol. 44: 603-617.
Stephan Ossowski, Rebecca Schwab, Detlef Weigel (2008) Gene silencing in plants using artificial microRNAs and other small RNAs. The Plant Journal 53 (4), 674-690.
Steppuhn, A., Gase, K., Krock, B., Halitschke, R., Baldwin, I. T. (2004) Nicotine's defensive function in nature. PLoS Biol. 2, 1074-1080.
Thines B., Katsir L., Melotto M., John Browse (2007) JAZ repressor proteins are targets of the SCFCOI1 complex during jasmonate signaling. Nature 448 (7154):661-665.
Todd, A. T. et al., (2010) A functional genomics screen identifies diverse transcription factors that regulate alkaloid biosynthesis in Nicotiana benthamiana. Plant J. 62, 589-600.
Voelckel, C., Krugel, T., Gase, K., Heidrich, N., van Dam, N. M., Winz, R., Baldwin, I. T. (2001) Antisense expression of putrescine N-methyltransferase confirms defensive role of nicotine in Nicotiana sylvestris against Manduca sexta. Chemoecology 11, 121-126.
Xu and Timko (2004) Methyl jasmonate induced expression of the tobacco putrescine N-methyltransferase genes requires both G-box and GCC-motif elements. Plant Molecular Biology 55: 743-761.
Wang, P., Liang, Z., Zeng, J., Li, W., Sun, X., Miao, Z., Tang, K. (2008) Generation of tobacco lines with widely different reduction in nicotine levels via RNA silencing approaches. J. Biosci. 33, 177-184.
Wang, P., Zeng, J., Liang, Z., Miao, Z., Sun, X., Tang, K. (2009) Silencing of PMT expression caused a surge of anatabine accumulation in tobacco. Mol. Biol. Rep. 36, 2285-2289.
Zhang F, Yao J, Ke J, Zhang L, Lam V Q, et al., (2015) Structural basis of JAZ repression of MYC transcription factors in jasmonate signaling. Nature 525 (7568):269-73.
Zhang, H-B. et al., (2012) Tobacco transcription factors NtMYC2a and NtMYC2b form nuclear complexes with the NtJAZ1 repressor and regulate multiple jasmonate-inducible steps in nicotine biosynthesis. Mol. Plant 5, 73-84.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All publicly available documents referenced or cited to herein, such as patents, patent applications, provisional applications, and publications, including GenBank Accession Numbers, are incorporated by reference in their entirety, including all figures and tables, to the extent they are not inconsistent with the explicit teachings of this specification.

Other embodiments are set forth within the following claims.

### SEQUENCE LISTING

| (684 bp) | |
|---|---|
| Open Reading Frame (ORF) of NtERF221 (XM_016622819): | |
| SEQ ID NO: 1 | |
| atgaatcccgctaatgcaaccttctctttctctgagcttgatttccttcaatcaatagaaaaccatcttct | |
| gaattatgattccgatttttctgaaattttttcgccgatgagttcaagtaacgcattgcctaatagtccta | |
| gctcaagttttggcagcttcccttcagcagaaaatagcttggatacctctctttgggatgaaaactttgag | |
| gaaacaatacaaaatctcgaagaaaagtccgagtccgaggaggaaacaaaggggcatgtcgtggcgcgtga | |
| gaaaaacgcgacacaagattggagacggtacataggagttaaacggcggccgtgggggacgttttcggcgg | |
| agataagggacccggagagaagaggcgcgagattatggctaggaacttacgagaccccagaggacgcagca | |
| ttggcttacgatcaagccgctttcaaaatccgcggctcgagagctcggctcaattttcctcacttaattgg | |
| atcaaacattcctaagccggctagagttacagcgagacgtagccgtacgcgctcaccccagccatcgtctt | |
| cttcatgtacctcatcatcagaaaatgggacaagaaaaaggaaaatagatttgataaattccatagccaaa | |
| gcaaaatttattcgtcatagctggaacctacaaatgttgctataa | |
| (378 bp) | |
| DNA Sequence of the 4 X GAG promoter: | |
| SEQ ID NO: 2 | |
| CTAACCCTGCACGTTGTAATGAATTTTTAACTATTATATTATATCGAGTTGCGCCCTC | |
| CACTTCTAGTCTAACCCTGCACGTTGTAATGAATTTTTAACTATTATATTATATCGA | |
| GTTGCGCCCTCCACTTCTAGTCTAACCCTGCACGTTGTAATGAATTTTTAACTATTAT | |
| ATTATATCGAGTTGCGCCCTCCACTTCTAGTCTAACCCTGCACGTTGTAATGAATTT | |
| TTAACTATTATATTATATCGAGTTGCGCCCTCCACTTCTAGAAATTG [symbol] TGC | |
| ATAGATGTTTATTGGGAGTGT [symbol] CAGCAATCTTTCGGAAAATACAAACCATAATACTT | |
| TCTCTTCTTCAATTTGTTTAGTTTAATTTTGAA | |
| BOLD = G-box | |
| ITALICS = GCC motif | |
| BOLD UNDERLINED = TATA box | |
| BOLD ITALICS UNDERLINED = Transcription start site | |
| (2046 bp) | |
| NtMYC1a ORF | |
| SEQ ID NO: 3 | |
| 1 | atgactgatt acagcttacc caccatgaat ttgtggaata ctagtggtac taccgatgac |
| 61 | aacgttacta tgatggaagc ttttatgtct tctgatctca cttcattttg ggctacttct |
| 121 | aattctactg ctgttgctgc tgttacctct aattctaatc atattccagt taatacccca |
| 181 | acggttcttc ttccgtcttc ttgtgcctct actgtcacag ctgtggctgt cgatgcttca |
| 241 | aaatccatgt cttttttcaa ccaagaaacc cttcaacagc gtcttcaaac gctcattgat |
| 301 | ggtgctcgtg agacgtggac ctatgccatc ttttggcagt catccgccgt tgatttaacg |
| 361 | agtccgtttg tgttgggctg gggagatggt tactacaaag gtgaagaaga taaagccaat |
| 421 | aggaaattag ctgtttcttc tcctgcttat atagctgagc aagaacaccg gaaaaaggtt |
| 481 | ctccgggagc tgaattcgtt gatttccggc acgcaaaccg gcactgatga tgccgtcgat |
| 541 | gaagaagtta ccgacactga atggttcttc cttatttcca tgacccagtc gtttgttaac |
| 601 | ggaagtgggc ttccgggtca ggccttatac aattccagcc ctatttgggt cgccggagca |
| 661 | gagaaattgg cagcttccca ctgcgaacgg gctcggcagg cccagggatt cgggcttcag |
| 721 | acgatggttt gtattccttc agcaaacggc gtggttgaat tgggctccac ggagttgatt |
| 781 | attcagagtt ctgatctcat gaacaaggtt agagtattgt ttaacttcaa taatgatttg |
| 841 | ggctctggtt cgtgggctgt gcaacccgag agcgatccgt ccgctctttg gctcactgat |
| 901 | ccatcgtctg cagctgtaca agtcaaagat ttaaatacag ttgaggcaaa ttcagttcca |
| 961 | tcaagtaata gtagtaagca agttgtattt gataatgaga ataatggtca cagttgtgat |
| 1021 | aatcagcaac agcaccattc tcggcaacaa acacaaggat tttttacaag ggagttgaac |
| 1081 | ttttcagaat tcgggtttga tggaagtagt aataatagga atgggaattc atcactttct |
| 1141 | tgcaagccag agtcggggga aatcttgaat tttggtgata gcactaagaa aagtgcaaat |
| 1201 | gggaacttat tttccggtca gtcccatttt ggtgcagggg aggagaataa gaagaagaaa |
| 1261 | aggtcacctg cttccagagg aagcaatgaa gaaggaatgc tttcatttgt ttcaggtaca |
| 1321 | atcttgcctg cagcttctgg tgcgatgaag tcaagtggat gtgtcggtga agactcctct |
| 1381 | gatcattcgg atcttgaggc ctcagtggtg aaagaagctg aaagtagtag agttgtagaa |
| 1441 | cccgaaaaga ggccaaagaa gcgaggaagg aagccagcaa atggacgtga ggaacctttg |
| 1501 | aatcacgtcg aagcagagag gcaaaggaga gagaaattaa accaaaggtt ctacgcttta |
| 1561 | agagctgttg ttccgaatgt gtccaagatg gacaaggcat cactgcttgg agatgcaatt |
| 1621 | tcatatatta atgagctgaa gttgaagctt caaactacag aaacagatag agaagacttg |
| 1681 | aagagccaaa tagaagattt gaagaaagaa ttagatagta aagactcaag gcgccctggt |
| 1741 | cctccaccac caaatcaaga tcacaagatg tctagccata ctggaagcaa gattgtagat |
| 1801 | gtggatatag atgttaagat aattggatgg gatgcgatga ttcgtataca atgtaataaa |
| 1861 | aagaaccatc cagctgcaag gttaatggta gccctcaagg agttagatct agatgtgcac |
| 1921 | catgccagtg tttcagtggt gaatgatttg atgatccaac aagccacagt gaaaatgggt |
| 1981 | agcagacttt acacggaaga gcaacttagg atagcattga catccagagt tgctgaaaca |
| 2041 | cgctaa |
| (681 AA) | |
| NtMYC1a polypeptide | |
| SEQ ID NO: 4 | |
| 1 | mtdyslptmn lwntsgttdd nvtmmeafms sdltsfwats nstavaavts nsnhipvntp |
| 61 | tvllpsscas tvtavavdas ksmsffnget lqqrlqtlid garetwtyai fwqssavdlt |
| 121 | spfvlgwgdg yykgeedkan rklavsspay iaeqehrkkv lrelnslisg tqtgtddavd |
| 181 | eevtdtewff lismtqsfvn gsglpgqaly nsspiwvaga eklaashcer arqaqgfglq |
| 241 | tmvcipsang vvelgsteli iqssdlmnkv rvlfnfnndl gsgswavqpe sdpsalwltd |
| 301 | pssaavqvkd lntveansvp ssnsskqvvf dnennghscd nqqqhhsrqq tqgfftreln |
| 361 | fsefgfdgss nnrngnssls ckpesgeiln fgdstkksan gnlfsgqshf gageenkkkk |
| 421 | rspasrgsne egmlsfvsgt ilpaasgamk ssgcvgedss dhsdleasvv keaessrvve |
| 481 | pekrpkkrgr kpangreepl nhveaerqrr eklnqrfyal ravvpnvskm dkasllgdai |
| 541 | syinelklkl qttetdredl ksqiedlkke ldskdsrrpg ppppnqdhkm sshtgskivd |
| 601 | vdidvkiigw damiriqcnk knhpaarlmv alkeldldvh hasvsvvndl miqqatvkmg |
| 661 | srlyteeqlr ialtsrvaet r |
| (2040 bp) | |
| NtMYC1b ORF | |
| SEQ ID NO: 5 | |
| 1 | cgcagacccc tcttttcacc catttctctc tctctctctc tctctctctc tatatatata |
| 61 | tatatctttc acgccaccat atccaactgt ttgtgctggg tttatggaat gactgattac |
| 121 | agcttaccca ccatgaattt gtggaatact agtggtacta ccgatgacaa cgtttctatg |
| 181 | atggaatctt ttatgtcttc tgatctcact tcattttggg ctacttctaa ttctactact |
| 241 | gctgctgtta cctctaattc taatcttatt ccagttaata ccctaactgt tcttcttccg |
| 301 | tcttcttgtg cttctactgt cacagctgtg gctgtcgatg cttcaaaatc catgtctttt |
| 361 | ttcaaccaag aaactcttca gcagcgtctt caaaccctca ttgatggtgc tcgtgagacg |
| 421 | tggacctatg ccatcttttg gcagtcatcc gtcgttgatt tatcgagtcc gtttgtgttg |
| 481 | ggctggggag atggttacta caaaggtgaa gaagataaag ccaataggaa attagctgtt |
| 541 | tcttctcctg cttatattgc tgagcaagaa caccgaaaaa aggttctccg ggagctgaat |
| 601 | tcgttgatct ccggcacgca aaccggcact gatgatgccg tcgatgaaga agttaccgac |
| 661 | actgaatggt tcttccttat ttccatgacc caatcgtttg ttaacggaag tgggcttccg |
| 721 | ggtcaggcct tatacaattc cagccctatt tgggtcgccg gagcagagaa attggcagct |
| 781 | tcccactgcg aacgggctcg gcaggcccag ggattcgggc ttcagacgat ggtttgtatt |
| 841 | ccttcagcaa acggcgtggt tgaattgggc tccacggagt tgataatcca gagttgtgat |
| 901 | ctcatgaaca aggttagagt attgtttaac ttcaataatg atttgggctc tggttcgtgg |
| 961 | gctgtgcagc ccgagagcga tccgtccgct ctttggctca ctgatccatc gtctgcagct |
| 1021 | gtagaagtcc aagatttaaa tacagttaag gcaaattcag ttccatcaag taatagtagt |
| 1081 | aagcaagttg tgtttgataa tgagaataat ggtcacagtt ctgataatca gcaacagcag |
| 1141 | cattctaagc atgaaacaca aggatttttc acaagggagt tgaatttttc agaatttggg |
| 1201 | tttgatggaa gtagtaataa taggaatggg aattcatcac tttcttgcaa gccagagtcg |
| 1261 | ggggaaatct tgaattttgg tgatagtact aagaaaagtg caaatgggaa cttattttcg |
| 1321 | ggtcagtccc attttggggc aggggaggag aataagaaca agaaaaggtc acctgcttcc |
| 1381 | agaggaagca atgaagaagg aatgctttca tttgtttcgg gtacaatctt gcctgcagct |
| 1441 | tctggtgcga tgaagtcaag tggaggtgta ggtgaagact ctgatcattc ggatcttgag |
| 1501 | gcctcagtgg tgaaagaagc tgaaagtagt agagttgtag aacccgaaaa gaggccaaag |
| 1561 | aagcgaggaa ggaagccagc aaatggacgg gaggaacctt tgaatcacgt cgaagcagag |
| 1621 | aggcaaagga gagagaaatt aaaccaaagg ttctacgcat taagagctgt tgttccgaat |
| 1681 | gtgtccaaga tggacaaggc atcactgctt ggagatgcaa tttcatatat taatgagctg |
| 1741 | aagttgaagc ttcaaaatac agaaacagat agagaagaat tgaagagcca aatagaagat |
| 1801 | ttaaagaaag aattagttag taaagactca aggcgccctg gtcctccacc atcaaatcat |
| 1861 | gatcacaaga tgtctagcca tactggaagc aagattgtag acgtggatat agatgttaag |
| 1921 | ataattggat gggatgcgat gattcgtata caatgtaata aaaagaatca tccagctgca |
| 1981 | aggttaatgg tagccctcaa ggagttagat ctagatgtgc accatgccag tgtttcagtg |
| 2041 | gtgaacgatt tgatgatcca acaagccact gtgaaaatgg gtagcagact ttacacggaa |
| 2101 | gagcaactta ggatagcatt gacatccaga gttgctgaaa cacgctaa |
| (679 AA) | |
| NtMYC1b polypeptide | |
| SEQ ID NO: 6 | |
| 1 | mtdyslptmn lwntsgttdd nvsmmesfms sdltsfwats nsttaavtsn snlipvntlt |
| 61 | vllpsscast vtavavdask smsffngetl qqrlqtlidg aretwtyaif wqssvvdlss |
| 121 | pfvlgwgdgy ykgeedkanr klavsspayi aeqehrkkvl relnslisgt qtgtddavde |
| 181 | evtdtewffl ismtqsfvng sglpgqalyn sspiwvagae klaashcera rqaqgfglqt |
| 241 | mvcipsangv velgstelii qscdlmnkvr vlfnfnndlg sgswavqpes dpsalwltdp |
| 301 | ssaavevqdl ntvkansvps snsskqvvfd nennghssdn qqqqhskhet qgfftrelnf |
| 361 | sefgfdgssn nrngnsslsc kpesgeilnf gdstkksang nlfsgqshfg ageenknkkr |
| 421 | spasrgsnee gmlsfvsgti lpaasgamks sggvgedsdh sdleasvvke aessrvvepe |
| 481 | krpkkrgrkp angreeplnh veaerqrrek lnqrfyalra vvpnvskmdk asllgdaisy |
| 541 | inelklklqn tetdreelks qiedlkkelv skdsrrpgpp psnhdhkmss htgskivdvd |
| 601 | idvkiigwda miriqcnkkn hpaarlmval keldldvhha svsvvndlmi qqatvkmgsr |
| 661 | lyteeqlria ltsrvaetr |
| (2214 bp) | |
| NtMYC2a gene | |
| SEQ ID NO: 7 | |
| CACACACTCTCTCCATTTTCACTCACTCCTTATCACCAAACAATTCTTGGGTGTTTGAATATAT | |
| ACCCGAAATAATTTCCTCTCTGTATCAAGAATCAAACAGATCTGAATTGATTTGTCTGTTTTTT | |
| TTTCTTGATTTTGTTATATGGAATGACGGATTATAGAATACCAACGATGACTAATATATGGAGC | |
| AATACTACATCCGATGATAATATGATGGAAGCTTTTTTATCTTCTGATCCGTCGTCGTTTTGGC | |
| CCGGAACAACTACTACACCAACTCCCCGGAGTTCAGTTTCTCCAGCGCCGGCGCCGGTGACGGG | |
| GATTGCCGGAGACCCATTAAAGTCTATGCCATATTTCAACCAAGAGTCACTGCAACAGCGACTC | |
| CAGACTTTAATCGATGGGGCTCGCAAAGGGTGGACGTATGCCATATTTTGGCAATCGTCTGTTG | |
| TGGATTTCGCGAGCCCCTCGGTTTTGGGGTGGGGAGATGGGTATTATAAAGGTGAAGAAGATAA | |
| AAATAAGCGTAAAACGGCGTCGTTTTCGCCTGACTTTATCACGGAACAAGCACACCGGAAAAAG | |
| GTTCTCCGGGAGCTGAATTCTTTAATTTCCGGCACACAAACCGGTGGTGAAAATGATGCTGTAG | |
| ATGAAGAAGTAACTGATACTGAATGGTTTTTTCTGATTTCCATGACACAATCGTTTGTTAACGG | |
| AAGCGGGCTTCCGGGCCTGGCGATGTATAGTTCAAGCCCGATTTGGGTTACTGGAACAGAGAGA | |
| TTAGCTGTTTCTCACTGTGAACGGGCCCGACAGGCCCAAGGTTTCGGGCTTCAGACTATTGTTT | |
| GTATTCCTTCAGCTAATGGTGTTGTTGAGCTCGGGTCAACTGAGTTGATATTCCAGACTGCTGA | |
| TTTAATGAACAAGGTTAAAGTTTTGTTTAATTTTAATATTGATATGGGTGCGACTACGGGCTCA | |
| GGATCGGGCTCATGTGCTATTCAGGCCGAGCCCGATCCTTCAGCCCTTTGGCTGACTGATCCGG | |
| CTTCTTCAGTTGTGGAAGTCAAGGATTCGTCGAATACAGTTCCTTCAAGGAATACCAGTAAGCA | |
| ACTTGTGTTTGGAAATGAGAATTCTGAAAATGGTAATCAAAATTCTCAGCAAACACAAGGATTT | |
| TTCACTAGGGAGTTGAATTTTTCCGAATATGGATTTGATGGAAGTAATACTCGGTATGGAAATG | |
| GGAATGCGAATTCTTCGCGTTCTTGCAAGCCTGAGTCTGGTGAAATCTTGAATTTTGGTGATAG | |
| TACTAAGAGGAGTGCTTGCAGTGCAAATGGGAGCTTGTTTTCGGGCCAATCACAGTTCGGGCCC | |
| GGGCCTGCGGAGGAGAACAAGAACAAGAACAAGAAAAGGTCACCTGCATCAAGAGGAAGCAACG | |
| ATGAAGGAATCCTTTCATTTGTTTCGGGTGTGATTTTGCCAAGTTCAAACACGGGGAAGTCCGG | |
| TGGAGGTGGCGATTCGGATCAATCAGATCTCGAGGCTTCGGTGGTGAAGGAGGCGGATAGTAGT | |
| AGAGTTGTAGACCCCGAGAAGAAGCCGAGGAAACGAGGGAGGAAACCGGCTAACGGGAGAGAGG | |
| AGCCATTGAATCATGTGGAGGCAGAGAGACAAAGGAGGGAGAAATTGAATCAAAGATTCTATGC | |
| ACTTAGAGCTGTTGTACCAAATGTGTCAAAAATGGATAAAGCATCACTTCTTGGTGATGCAATT | |
| GCATTTATCAATGAGTTGAAATCAAAGGTTCAGAATTCTGACTCAGATAAAGAGGACTTGAGGA | |
| ACCAAATCGAATCTTTAAGGAATGAATTAGCCAACAAGGGATCAAACTATACCGGTCCTCCCCC | |
| GTCAAATCAAGAACTCAAGATTGTAGATATGGACATCGACGTTAAGGTGATCGGATGGGATGCT | |
| ATGATTCGTATACAATCTAATAAAAAGAACCATCCAGCCGCGAGGTTAATGACCGCTCTCATGG | |
| AATTGGACTTAGATGTGCACCATGCTAGTGTTTCAGTTGTCAACGAGTTGATGATCCAACAAGC | |
| GACTGTGAAAATGGGAAGCCGGCTTTACACGCAAGAACAACTTCGGATATCATTGACATCCAGA | |
| ATTGCTGAATCGCGATGAAGAGAAATACAGTAAATGGAAATTATCATAGTGAGCTCTGAATAAT | |
| GTTATCTTTCATTGAGCTATTTTAAGAGAATTTCTCCTAAAAAAAAAAAAAAAAAAAAAAAAAA | |
| A | |
| (659 AA) | |
| NtMYC2a polypeptide | |
| SEQ ID NO: 8 | |
| 1 | mtdyriptmt niwsnttsdd nmmeaflssd pssfwpgttt tptprssysp apapvtgiag |
| 61 | dplksmpyfn geslqqrlqt lidgarkgwt yaifwqssvv dfaspsvlgw gdgyykgeed |
| 121 | knkrktasfs pdfiteqahr kkvlrelnsl isgtqtggen davdeevtdt ewfflismtq |
| 181 | sfvngsglpg lamyssspiw vtgterlavs hcerarqaqg fglqtivcip sangvvelgs |
| 241 | telifqtadl mnkvkvlfnf nidmgattgs gsgscaiqae pdpsalwltd passvvevkd |
| 301 | ssntvpsrnt skqlvfgnen senvnqnsqq tqgfftreln fseygfdgsn trygngnans |
| 361 | srsckpesge ilnfgdstkr sacsangslf sgqsqfgpgp aeenknknkk rspasrgsnd |
| 421 | egilsfvsgv ilpssntgks ggggdsdqsd leasvvkead ssrvvdpekk prkrgrkpan |
| 481 | greeplnhve aerqrrekln qrfyalravv pnvskmdkas llgdaiafin elkskvqnsd |
| 541 | sdkedlrnqi eslrnelank gsnytgppps nqelkivdmd idvkvigwda miriqsnkkn |
| 601 | hpaarlmtal meldldvhha svsvvnelmi qqatvkmgsr lytgeglris ltsriaesr |
| (2391 bp) | |
| NtMYC2b gene | |
| SEQ ID NO: 9 | |
| GTAACAAACCCTCTCCATTTTCACTCACTCCAAAAAACTTTCCTCTCTATTTTTTCTCTCTGTA | |
| TCAAGAATCAAACAGATCTGAATTGATTTGGGAGTTTTTTTTCTTCTTGTTTTTGTTATATGGA | |
| ATGACGGACTATAGAATACCAACGATGACTAATATATGGAGCAATACAACATCCGACGATAACA | |
| TGATGGAAGCTTTTTTATCTTCTGATCCGTCGTCGTTTTGGGCCGGAACAAATACACCAACTCC | |
| ACGGAGTTCAGTTTCTCCGGCGCCGGCGCCGGTGACGGGGATTGCCGGAGACCCATTAAAGTCG | |
| ATGCCGTATTTCAACCAAGAGTCGCTGCAACAGCGACTCCAGACGTTAATCGACGGGGCTCGCG | |
| AAGCGTGGACTTACGCCATATTCTGGCAATCGTCTGTTGTGGATTTCGTGAGCCCCTCGGTGTT | |
| GGGGTGGGGAGATGGATATTATAAAGGAGAAGAAGACAAGAATAAGCGTAAAACGGCGGCGTTT | |
| TCGCCTGATTTTATTACGGAGCAAGAACACCGGAAAAAAGTTCTCCGGGAGCTGAATTCTTTAA | |
| TTTCCGGCACACAAACTGGTGGTGAAAATGATGCTGTAGATGAAGAAGTAACGGATACTGAATG | |
| GTTTTTTCTGATTTCAATGACTCAATCGTTTGTTAACGGAAGCGGGCTTCCGGGCCTGGCTATG | |
| TACAGCTCAAGCCCGATTTGGGTTACTGGAAGAGAAAGATTAGCTGCTTCTCACTGTGAACGGG | |
| CCCGACAGGCCCAAGGTTTCGGGCTTCAGACTATGGTTTGTATTCCTTCAGCTAATGGTGTTGT | |
| TGAGCTCGGGTCAACTGAGTTGATATTCCAGAGCGCTGATTTAATGAACAAGGTTAAAATCTTG | |
| TTTGATTTTAATATTGATATGGGCGCGACTACGGGCTCAGGTTCGGGCTCATGTGCTATTCAGG | |
| CTGAGCCCGATCCTTCAACCCTTTGGCTTACGGATCCACCTTCCTCAGTTGTGGAAGTCAAGGA | |
| TTCGTCGAATACAGTTCCTTCAAGTAATAGTAGTAAGCAACTTGTGTTTGGAAATGAGAATTCT | |
| GAAAATGTTAATCAAAATTCTCAGCAAACACAAGGATTTTTCACTAGGGAGTTGAATTTTTCCG | |
| AATATGGATTTGATGGAAGTAATACTAGGAGTGGAAATGGGAATGTGAATTCTTCGCGTTCTTG | |
| CAAGCCTAGAAATGCTTCAAGTGCAAATGGGAGCTTGTTTTCGGGCCAATCGCAGTTCGGTCCC | |
| GGGCCTGCGGAGGAGAACAAGAACAAGAACAAGAAAAGGTCACCTGCATCAAGAGGAAGCAATG | |
| AAGAAGGAATGCTTTCATTTGTTTCGGGTGTGATCTTGCCAAGTTCAAACACGGGGAAGTCCGG | |
| TGGAGGTGGCGATTCGGATCATTCAGATCTCGAGGCTTCGGTGGTGAAGGAGGCGGATAGTAGT | |
| AGAGTTGTAGACCCCGAGAAGAGGCCGAGGAAACGAGGAAGGAAACCGGCTAACGGGAGAGAGG | |
| AGCCATTGAATCATGTGGAGGCAGAGAGGCAAAGGAGGGAGAAATTGAATCAAAGATTCTATGC | |
| ACTTAGAGCTGTTGTACCAAATGTGTCAAAAATGGATAAAGCATCACTTCTTGGTGATGCAATT | |
| GCATTTATCAATGAGTTGAAATCAAAGGTTCAGAATTCTGACTCAGATAAAGATGAGTTGAGGA | |
| ACCAAATTGAATCTTTAAGGAATGAATTAGCCAACAAGGGATCAAACTATACCGGTCCTCCACC | |
| GCCAAATCAAGATCTCAAGATTGTAGATATGGATATCGACGTTAAAGTCATCGGATGGGATGCT | |
| ATGATTCGTATACAATCTAATAAAAAGAACCATCCAGCCGCGAGGTTAATGGCCGCTCTCATGG | |
| AATTGGACTTAGATGTGCACCATGCTAGTGTTTCAGTTGTCAACGAGTTGATGATCCAACAAGC | |
| GACAGTGAAAATGGGGAGCCGGCTTTACACGCAAGAGCAGCTTCGGATATCATTGACATCCAGA | |
| ATTGCTGAATCGCGATGAAGAGAAATACAGTAAATGGAAATTATTAGTGAGCTCTGAATAATGT | |
| TATCTTTCATTGAGCTATTTTAAGAGAATTTCTCCTATAGTTAGATCTTGAGATTAAGGCTACT | |
| TAAAAGTGGAAAGTTGATTGAGCTTTCCTCTTAGTTTTTTGGGTATTTTTCAACTTTTATATCT | |
| AGTTTGTTTTCCACATTTTCTGTACATATAATGTGAAACCAATACTAGATCTCAAGATCTGGTT | |
| TTTAGTTCTGTAATTAGAAATAAATATGCAGCTTCATCTTTTTCTGTTAAAAAAAAAAAAAAAA | |
| AAAAAAAAA | |
| (658 AA) | |
| NtMYC2b polypeptide | |
| SEQ ID NO: 10 | |
| 1 | mtdyriptmt niwsnttsdd nmmeaflssd pssfwagtnt ptprssvspa papvtgiagd |
| 61 | plksmpyfnq eslqqrlqtl idgareawty aifwqssvvd fvspsvlgwg dgyykgeedk |
| 121 | nkrktaafsp dfiteqehrk kvlrelnsli sgtqtggend avdeevtdte wfflismtqs |
| 181 | fvngsglpgl amyssspiwv tgrerlaash cerarqaqgf glqtmvcips angvvelgst |
| 241 | elifqsadlm nkvkilfdfn idmgattgsg sgscaiqaep dpstlwltdp pssvvevkds |
| 301 | sntvpssnss kqlvfgnens envnqnsqqt qgfftrelnf seygfdgsnt rsgngnvnss |
| 361 | rsckpesgei lnfgdstkrn assangslfs gqsqfgpgpa eenknknkkr spasrgsnee |
| 421 | gmlsfvsgvi lpssntgksg gggdsdhsdl easvvkeads srvvdpekrp rkrgrkpang |
| 481 | reeplnhvea erqrreklnq rfyalravvp nvskmdkasl lgdaiafine lkskvqnsds |
| 541 | dkdelrnqie slrnelankg snytgppppn qdlkivdmdi dvkvigwdam iriqsnkknh |
| 601 | paarlmaalm eldldvhhas vsvvnelmiq qatvkmgsrl ytqeqlrisl tsriaesr |

1. A Nicotiana plant comprising a chimeric nucleic acid construct comprising a nucleotide sequence overexpressing a gene product encoded by NtERF221 operably linked to a heterologous promoter such that NtERF221 is overexpressed relative to a wild-type control plant, whereby the Nicotiana plant accumulates commercial levels of nicotine in its leaves without topping, wherein the nucleotide sequence is selected from the group consisting of:
(a) a nucleotide sequence set forth in SEQ ID NO: 1; and
(b) a nucleotide sequence that is at least about 90% identical to the nucleotide sequence of (a), and which encodes an NtERF221 transcription factor that positively regulates nicotine biosynthesis.

2. The Nicotiana plant of claim 1, wherein the heterologous promoter is selected from the group consisting of a dual CaMV 35S promoter, a Glycine Max Ubiquitin 3 (GmUBI3) gene promoter, and a jasmonate-inducible promoter having the nucleotide sequence set forth in SEQ ID NO: 2.

3. The Nicotiana plant of claim 2, wherein the heterologous promoter is the jasmonate-inducible promoter having the nucleotide sequence set forth in SEQ ID NO: 2

4. The Nicotiana plant of claim 1, wherein the plant is a Nicotiana tabacum plant.

5. Seeds from the plant of claim 1, wherein the seeds comprise the chimeric nucleic acid construct.

6. A tobacco product comprising the Nicotiana plant of claim 1, wherein the product has an increased level of nicotine as compared to a tobacco product from a wild-type control plant.

7. The tobacco plant of claim 1, wherein the commercial level of nicotine in the tobacco leaves is in the range from about 2.5% to about 6%.

8. A population of tobacco plants characterized by homozygosity for a nucleotide sequence overexpressing a gene product encoded by NtERF221, wherein expression of the gene product is driven by a heterologous promoter such that NtERF221 is overexpressed as compared to a wild-type control tobacco plant, whereby the population stably displays a phenotype comprising a commercial level of nicotine in the tobacco plant leaves without topping, wherein the nucleotide sequence is selected from the group consisting of:
(a) a nucleotide sequence set forth in SEQ ID NO: 1; and
(b) a nucleotide sequence that is at least about 90% identical to the nucleotide sequence of (a), and which encodes an NtERF221 transcription factor that positively regulates nicotine biosynthesis.

9. The population of claim 8, wherein the commercial level of nicotine in the tobacco leaves is in the range from about 2.5% to about 6%.

10. The population of claim 8, wherein the heterologous promoter is selected from the group consisting of a dual CaMV 35S promoter, a Glycine Max Ubiquitin 3 (GmUBI3) gene promoter, and a jasmonate-inducible promoter having the nucleotide sequence set forth in SEQ ID NO: 2.

11. The population of claim 10, wherein the heterologous promoter is the jasmonate-inducible promoter having the nucleotide sequence set forth in SEQ ID NO: 2

12. The population of claim 8, wherein the plants are Nicotiana tabacum plants.

13. Seeds from the population of claim 8, wherein the seeds comprise the chimeric nucleic acid construct.

14. A tobacco product comprising the population of tobacco plants of claim 8, wherein the product has an increased level of nicotine as compared to a tobacco product from wild-type control plants.

15. A method for increasing nicotine in a Nicotiana plant, comprising:
(a) introducing into the Nicotiana plant an expression vector comprising a heterologous promoter operably linked to a nucleotide sequence selected from the group consisting of:
(i) a nucleotide sequence set forth in SEQ ID NO: 1; and
(ii) a nucleotide sequence that is at least about 90% identical to the nucleotide sequence of (i), and which encodes a transcription factor that positively regulates nicotine biosynthesis; and
(b) growing the plant under conditions that allow for the expression of a transcription factor that positively regulates nicotine biosynthesis from the nucleotide sequence;
wherein expression of the transcription factor results in the plant having an increased nicotine content as compared to a wild-type control plant grown under similar conditions.

16. The method of claim 15, wherein the heterologous promoter is selected from the group consisting of a dual CaMV 35S promoter, a Glycine Max Ubiquitin 3 (GmUBI3) gene promoter, and a jasmonate-inducible promoter having the nucleotide sequence set forth in SEQ ID NO: 2.

17. The method of claim 16, wherein the heterologous promoter is the jasmonate-inducible promoter having the nucleotide sequence set forth in SEQ ID NO: 2.

18. The method of claim 15, further comprising overexpressing within the Nicotiana plant at least one of NBB1, A622, quinolate phosphoribosyltransferase (QPT), putrescine N-methyltransferase (PMT), ornithine decarboxylase (ODC), aspartate oxidase (AO), quinolinic acid synthase (QS), or N-methylputrescine oxidase (MPO).

19. The method of claim 15, further comprising overexpressing within the Nicotiana plant at least one additional transcription factor that positively regulates nicotine biosynthesis.

20. The method of claim 19, wherein the additional transcription factor that positively regulates nicotinic alkaloid biosynthesis is at least one of NtMYC1a, NtMYC1b, NtMYC2a, or NtMYC2b.

21. The method of claim 15, wherein the vector comprises the nucleotide sequence set forth in SEQ ID NO: 2.

22. The method of claim 15, further comprising topping the tobacco plant and/or treating the plant with exogenous jasmonic acid.
